(19) United States
(12) Patent Application Publication
(10) Pub. No.: US 2014/0350080 A1
(43) Pub. Date: Nov. 27, 2014

(54) INHIBITION OF VIRAL GENE EXPRESSION

(71) Applicants: UNIVERSITY OF THE WITWATERSRAND, JOHANNESBURG, Johannesburg (ZA); GOETHE-UNIVERSITY, Frankfurt am Main (DE)

(72) Inventors: Patrick Arbuthnot, Johannesburg (ZA); Justin Hean, Johannesburg (ZA); Abdullah Ely, Johannesburg (ZA); Musa Marimani, Johannesburg (ZA); Jolanta Brzezinska, Frankfurt am Main (DE); Jennifer D'Onofrio, Frankfurt am Main (DE); Maximilian C.R. Buff, Frankfurt am Main (DE); Joachim W. Engels, Frankfurt am Main (DE); Stefan Bernhardt, Frankfurt am Main (DE)

(73) Assignee: UNIVERSITY OF THE WITWATERSRAND, JOHANNESBURG, Johannesburg (ZA)

(21) Appl. No.: 14/354,180

(22) PCT Filed: Oct. 26, 2012
(86) PCT No.: PCT/IB2012/055915

§ 371 Date: Apr. 25, 2014

(30) Foreign Application Priority Data
Oct. 28, 2011 (ZA) 2011/07890

(51) Int. Cl. C12N 15/113

(52) U.S. Cl. CPC C12N 15/1131; C12N 2320/30
USPC 514/44 A; 536/24.5

(57) ABSTRACT

This invention relates to modified short interfering RNA (siRNA) nucleic acid molecules, particularly siRNA's which have been modified by the addition of a 2-0-guanidinopropyl (GP) modified nucleoside. In particular the invention relates to modified siRNAs which are capable of silencing target sequences, methods of treating and preventing infection by using the siRNAs, medicaments containing the siRNAs and use of the siRNAs.

### INTRODUCTION

The present invention relates to modified short interfering RNA (siRNA) molecules that modulate the expression of genes via the RNA interference pathway. The nucleic acid molecules encoding the siRNAs of the invention include one or more modifications which produce differences in their physical properties when compared to wild type, unmodified siRNAs. In a preferred embodiment of the invention the nucleic acid sequences of the siRNAs include at least one nucleoside having a 2′-O-guanidinopropyl (GP) moiety. In further embodiments of the invention the modification of the siRNA results in enhanced stability of the modified siRNA, improved gene silencing by the modified siRNA and attenuated immunostimulation.

### BACKGROUND OF THE INVENTION

Synthetic RNAi activators have shown considerable potential for therapeutic application to silencing of pathology-causing genes. Typically these exogenous RNAi activators comprise duplex RNA of approximately 21 bp with 2 nt overhangs at the 3′ ends. To improve efficacy of siRNAs, chemical modification at the 2′-OH group of ribose has been employed. Enhanced stability, gene silencing and attenuated immunostimulation have been demonstrated using this approach. Although promising, efficient and controlled delivery of highly negatively charged nucleic acid gene silencers remains problematic.

To assess the potential utility of introducing positively charged groups at the 2′ position, our investigations aimed at assessing efficacy of novel siRNAs containing 2′-O-guanidinopropyl (GP) moieties. We describe the formation of all four GP-modified nucleosides using the synthesis sequence of Michael addition with acrylonitrile followed by Raney-Ni reduction and guanidinylation. These precursors were used successfully to generate anti-hepatitis B virus (HBV) siRNAs. Testing in a cell culture model of viral replication demonstrated that the GP modifications improved silencing. Moreover, thermodynamic stability was not affected by the GP moieties and their introduction into each position of the seed region of the siRNA guide strand did not alter the silencing efficacy of the intended HBV target. These results demonstrate that modification of siRNAs with GP groups confers properties that may be useful for advancing therapeutic application of synthetic RNAi activators.

Use of synthetic small interfering RNAs (siRNAs) to trigger RNA interference- (RNAi-) mediated gene silencing has shown considerable potential for therapeutic application [1], [2], [3]. Typically, siRNAs are synthetic mimics of natural Dicer products and comprise 21-25 nucleotide (nt) duplexes with 2 nt 3′ overhangs. Progress with use of synthetic siRNAs has profited from vast experience gained from developing antisense RNA molecules. Consequently advances have been rapid and improving siRNA efficacy has benefited from valuable biological and synthetic chemistry insights. Advantages of synthetic siRNAs over expressed RNAi activators are that they are amenable to chemical modification to improve stability, safety and specificity [4], [5]. Also, controlled large scale preparation necessary for clinical use is feasible with chemical synthetic procedures. Nevertheless, despite significant advances, the delivery of these polyanionic nucleic acids across lipid-rich cell membranes remains problematic. Vectors used to transport synthetic RNAi activators to target cells have included cationic lipid-containing lipoplexes [6], conjugations to peptides [7] or oligocationic compounds such as spermidine [8]. However, success using these methods has been variable. To overcome difficulties of the excessive negative charge of nucleic acids, while at the same time improving thermal and serum stability, we previously investigated an approach that entailed 2′-modification of ribose with cationic groups [9], [10]. Initially we generated always 2′-O-aminoethyl-adenosine and 2′-O-aminoethyl uridine. Synthesis entailed initial alkylation by methyl bromoacetate, which was followed by a series of transformation reactions. Using a luciferase reporter assay to measure knockdown, it was demonstrated that the 2′-O-aminoethyl modifications were at least as efficient as 2′-OMe siRNA modifications. An important property of the 2′-O-aminoethyl derivatives was their ability to rescue less active siRNAs when the chemical modifications were placed at the 3′ end of the siRNA passenger strand [11]. Subsequently this approach was advanced by developing methods that enabled successful alkylation of all four ribonucleosides [12]. This was achieved using phalimidoethyltriflate as an alkylating agent and with this methodology all four phosphoramidites bearing 2′-O-aminoethyl side chains were formed. Although encouraging, a problem of using these siRNA reagents is that the yields of the multistep chemical synthesis are typically low. Moreover scaling up the synthesis reaction is difficult.

To address these concerns, we have investigated utility, of an alternative 2′-O-guanidinopropyl (GP) nucleoside modification method. Using the novel approach reported here, we describe the formation of all four GP-modified nucleosides using the synthesis sequence of Michael addition with acrylonitrile [13, 14, 15] followed by Raney-Ni reduction [16] and guanidinylation. Efficiency of the GP siRNAs was assessed in a cell culture model of hepatitis B virus (HBV) replication using target sequences that have previously been shown to be suitable for RNAi-based inhibition of viral replication [17, 18, 19, 20]. Results demonstrate more effective silencing of markers of viral replication than unmodified counterparts. Moreover, the GP-modified siRNAs were more stable to serum conditions than the unmodified controls.

### SUMMARY OF THE INVENTION

The present invention provides modified nucleic acid molecules and compositions comprising the modified nucleic acid molecules.

According to a first aspect of the invention the modified nucleic acid molecules comprise modified short interfering RNA (siRNA) nucleic acid molecules. The modified siRNA molecules comprise a sense strand and an antisense strand, and at least one nucleotide in the sense strand or at least one nucleotide in the antisense strand which is derived from a 2′-O-guanidinopropyl (GP) modified nucleoside. Further, the nucleic acid molecule is capable of silencing the expression of a target sequence wherein the target sequence is a DNA or RNA sequence.

The present invention teaches that at least one of the modified nucleosides is selected from the group consisting of a 2′-O-guanidinopropyl adenosine phosphoramidite, a 2′-O-guanidinopropyl cytidine phosphoramidite, a 2′-O-guanidinopropyl guanosine phosphoramidite and a 2′-O-guanidinopropyl uridine phosphoramidite. Further the invention provides for siRNAs containing combinations of the aforementioned phosphoramidites.

Preferably, the sense and antisense strands of the modified nucleic acid molecule are each, independently 18 to 26 nucleotides in length, preferably 19 to 25 nucleotides in length and most preferably 21 nucleotides in length.

Preferably, the at least one modified nucleotide may be located in the sense or the antisense stand or both. Further, the sense and antisense strands of the modified nucleic acid molecule will preferably both comprise artificially synthesised sequences.

It will be appreciated that the modified siRNA, may include an siRNA which targets DNA or RNA from any organism, including microorganisms, plants or animals. Preferably, the siRNA will target complementary nucleic acid molecules in microorganisms, including bacteria and viruses. More preferably the siRNA will target complementary nucleic acid sequence of a virus.

It will further be appreciated that the modified siRNA of the invention is a nucleic acid molecule.

In a preferred embodiment of the invention the modified siRNA inhibits viral replication. Preferably, the virus is a hepatitis virus and most preferably the virus is a hepatitis B virus.

The modified siRNA of the invention does not induce a detectable interferon response compared to an unmodified siRNA when transfected into cultured cells and/or in in vivo applications. Further, the modified siRNA has greater stability in a standard serum assay than an unmodified siRNA comprising the same sequence. In a further embodiment the modified siRNA exhibits greater knockdown of target gene expression than an unmodified siRNA comprising the same sequence.

In a preferred embodiment of the invention the antisense strand may comprise a sequence of SEQ ID NO: 1. Further the at least one 2′-O-guanidinopropyl (GP) modified nucleoside may be inserted at position 2, 3, 4, 5, 6, 7, 8, 9, 10, 11, 12, 13, 14, 15, 16, 17, 18, 19, 20 and/or 21 of the antisense strand or at any combination of these positions.

The antisense strand may comprise an unmodified sequence of SEQ ID NO: 1 or any one of the sequences set forth in SEQ ID NOs: 5, 6, 7, 8, 9, 10, 11, 12, 13, 14, 15, 16, 17, 18, 19, 20, 21, 22, 23, 24, 25, 26, 27, 28, 29 or 30.

In another preferred embodiment of the invention the sense strand may comprise a sequence of SEQ ID NO: 2. Further, at least one 2′-O-guanidinopropyl (GP) modified nucleoside has been inserted at position 2, 3, 4, 5, 6, 7, 8, 9, 10, 11, 12, 13, 14, 15, 16, 17, 18, 19, and/or 21 of the sense strand or at any combination of these positions.

The sense strand may comprise an unmodified sequence of SEQ ID NOs: 2 or any one of the sequences set forth in SEQ ID NOs: 31 or 32.

According to a second aspect of the present invention there is provided for a method of treatment or prevention of viral infection, wherein the method comprises administering a therapeutically amount of the nucleic acid molecule of the invention together with and a pharmaceutically acceptable adjuvant and/or carrier to a subject in need thereof. The subject may be an animal, preferably a mammal and most preferably a human. Further, the viral infection may be hepatitis infection and most preferably the hepatitis infection is hepatitis B.

According to a third aspect of the present invention there is provided for the use of the modified siRNA of the invention in the treatment or prevention of viral infection, wherein the method comprises administering a therapeutically amount of the nucleic acid molecule of the invention together with and a pharmaceutically acceptable adjuvant and/or carrier to a subject in need thereof. The subject may be an animal, preferably a mammal and most preferably a human. Further, the viral infection may be hepatitis virus infection and most preferably the hepatitis virus infection is caused by hepatitis B.

According to a fourth aspect of the present invention there is provided for the manufacture of a medicament for use in a method of treatment or prevention of viral infection, wherein the method comprises administering a therapeutically amount of the nucleic acid molecule of the invention together with and a pharmaceutically acceptable adjuvant and/or carrier to a subject in need thereof. The subject may be an animal, preferably a mammal and most preferably a human. Further, the viral infection may be hepatitis virus infection and most preferably the hepatitis virus infection is caused by hepatitis B.

In a further aspect of the invention, there is provided for a composition comprising the siRNA of the invention together with pharmaceutically acceptable excipients, carriers, adjuvants and the like. Further, there is provided for a kit comprising the aforementioned composition together with instructions for use of the composition.

### BRIEF DESCRIPTION OF THE FIGURES

Non-limiting embodiments of the invention will now be described by way of example only and with reference to the following figures:

FIG. 1 shows synthesis of the 2′-O-guanidinopropyl adenosine-, cytidine- and uridine-phosphoramidites required for oligoribonucleotide preparation. (i) acrylonitrile, CsCO₃, tert-butyl alcohol, rt; (ii) H₂N—NH₂.H₂O, methanol, rt (adenosine and cytidine derivative); no deprotection of the uridine derivative; (iii) H₂ (30 bar), NH₃, methanol, 30-60 min, rt; (iv) N,N′-di-Boc-N″-triflylguanidine, Et₃N, CH₂Cl₂, 0° C. (30 min) to it (30 min); (v) DMF-dimethyl diacetale, methanol, rt (adenosine derivative); benzoyl chloride, pyridine, 0° C. (30 min) to it (30 min) (cytidine derivative); no protection group was applied to the uridine derivative; (vi) Et₃N.3HF, THF, rt; (vii) 4,4′-dimethoxytrityl chloride, pyridine, rt; (viii) 2-cyanoethyl N,N,N′N′-tetraisopropyl phosphane, 4,5-dicyanoimidazole, CH₂Cl₂, rt.

FIG. 2 shows synthesis of the 2′-O-guanidinopropyl guanosine phosphoramidite required for oligoribonucleotide preparation. (i) acrylonitrile, CsCO₃, tert-butyl alcohol, rt; (ii) formic acid (70%), dioxane/water; (iii) H₂ (30 bar), NH₃, methanol, 30-60 min, rt; (iv) N,N′-di-Boc-N″-triflylguanidine, Et₃N, CH₂Cl₂, 0° C. (30 min) to it (30 min); (v) isobutyryl chloride, pyridine, 0° C. (1 h) to it (1 h); (vi) Et₃N.3HF, THF, rt; (vii) 4,4′-dimethoxytrityl chloride, pyridine, rt; (viii) 2-cyanoethyl N,N,N′,N′-tetraisopropyl phosphane, 4,5-dicyanoimidazole, CH₂Cl₂, rt.

FIG. 3 shows the improved method of synthesis of the 2′-O-guanidinopropyl-N²-dmf-guanosine phosphoramidite for oligoribonucleotide synthesis. (i) acrylonitrile, Cs₂CO₃, tert-butyl alcohol, rt; (ii) formic acid (70%), dioxane/water; (iii) H₂ (30 bar), NH₃, methanol, 30-60 min, rt; (iv) N,N′-di-Boc-N″-triflylguanidine, Et₃N, CH₂Cl₂, 0° C. (30 min) to it (30 min); (v) N,N-dimethylformamide dimethyl acetal, methanol, it (12 h); (vi) Et₃N.3HF, THF, rt; (vii) 4,4′-dimethoxytrityl chloride, pyridine, rt; (viii) 2-cyanoethyl N,N,N′,N′-tetraisopropyl phosphane, 4,5-dicyanoimidazole, CH₂Cl₂, rt.

FIG. 4 shows organisation of the hepatitis B virus genome and indicates the site targeted by the antiHBV siRNA3 used in this study. Nucleotide co-ordinates of the genome are given relative to the single EcoRI restriction site (HBV genotype A, GenBank: AP007263.1). The sequence targeted by HBV siRNA3 extends from nucleotide 1693 to 1711. Partially double-stranded HBV DNA comprises + and − strands with cohesive complementary 5′ ends. The cis-elements that regulate HBV transcription are represented by the circular and rectangular symbols. Immediately surrounding arrows indicate the viral open reading frames (with initiation codons) that encompass the entire genome. Four outer arrows indicate the HBV transcripts, which have common 3′ ends that all include HBx.

FIG. 5 shows dual luciferase assay to determine efficacy of 2′-O-guanidinopropyl-modified antiHBV siRNAs. A. Schematic illustration of dual luciferase reporter plasmid. The HBx target sequence was inserted downstream of the hRLuc ORF. Renilla luciferase activity was used as an indicator of target silencing and efficacy was determined relative to activity of constitutively expressed Firefly luciferase. B. Ratio of Renilla to Firefly luciferase activity following cotransfection with indicated siRNAs together with dual luciferase reporter plasmid. Controls included a mock transfection in which inert plasmid DNA was substituted for siRNA as well as a scrambled siRNA that did not have complementary sequences to the HBx target. Data are represented as mean ratios of Renilla to Firefly luciferase activity (±SEM) and are normalised relative to the mock treated cells. Differences were considered statistical significant when the p value, determined according to the Student's 2 tailed paired t-test, was less than 0.05.

FIG. 6 shows inhibition of HBV replication by antiHBV siRNAs in cultured cells. A. Illustration of the HBV replication competent plasmid, pCH-9/3091, together with site targeted by HBV siRNA3. pCH-9/3091 was used to transfect liver-derived Huh7 cells in culture. B. The concentration of HBsAg was measured in cell culture supernatants following cotransfection 2′-O-guanidinopropyl-modified siRNAs. Values are given as relative optical density (OD) readings from the ELISA assay. Unmodified siRNA did not include 2′-O-Guanidinopropyl residues. The control was a scrambled siRNA that did not have complementary sequences to the HBx target. Data are represented as mean relative concentrations of HBsAg (OD±SEM) and are normalised relative to the mock treated cells. Differences were considered statistical significant when the p value, determined according to the Student's 2 tailed paired t-test, was less than 0.05.

FIG. 7 shows assessment of stability of 2′-O-guanidinopropyl-modified siRNAs. The panel of 2′-O-guanidinopropyl-modified siRNAs was incubated with DMEM alone, or DMEM with 80% fetal calf serum, for times ranging from 0 to 24 hours. Thereafter degradation of siRNAs was assessed using polyacrylamide gel electrophoresis with ethidium bromide staining.

FIG. 8 shows assessment of interferon response in transfected HEK293 cells. Cells were transfected with the indicated siRNAs, or with poly (I:C). RNA was extracted from the cells 24 hours later and then subjected to quantitative real time PCR to determine concentrations of IFN-β and GAPDH mRNA. Means (±SEM) of the normalised ratios of IFN-β to GAPDH mRNA concentrations are indicated from 3 independent experiments. The poly (I:C) positive control verified that an interferon response was induced in the cells under the conditions used here.

FIG. 9 shows the assessment of toxicity in cells that had been transfected with the indicated unmodified and modified siRNAs. Toxicity of siRNAs in vitro was assessed by performing the MTT (3-(4,5-Dimethylthiazol-2-yl)-2,5-diphenyltetrazolium bromide) assay. Cells were either transfected with modified siRNAs (experimental) or unmodified siRNAs or were untransfected (controls). Data was analysed after quantifying the ratios of the optical densities at 570 nm (product) to the optical density at 655 nm (indicator of cell number). The data shows that there was no significant difference between transfected and untransfected cells, which demonstrates that the tested siRNAs did not display a toxicological profile in vitro. Values represent the means±standard deviation of 3 replicate transfections (*p<0.05).

FIG. 10 shows a schematic illustration of partial and complete HBV targets incorporated into the dual luciferase reporter constructs. The HBx target sequences comprising the complete target (A), Incomplete Target 1 (IT1) (B), Incomplete Target 2 (IT2) (C) and Seed Only (SO) (D) were inserted downstream of the hRLuc ORF. Renilla luciferase activity was used as an indicator of target silencing and efficacy was determined relative to activity of constitutively expressed Firefly luciferase. These reporter plasmids were used to compare the effect of the position of 2′-O-guanidinopropyl-modified anti-HBV siRNAs on the silencing of perfectly complementary and incomplete HBV targets.

FIG. 11 shows the ratio of Renilla to Firefly luciferase activity following cotransfection with indicated siRNAs together with dual luciferase reporter plasmids incorporating complete (CT), incomplete 1 (IT1), incomplete 2 (IT2) and seed only (SO) HBV target sequences. Controls included a mock transfection in which inert plasmid DNA was substituted for siRNA as well as a scrambled siRNA that did not have complementary sequences to the HBx target. Experiments were performed in triplicate and performed in batches where modified siRNAs included the GP groups at positions 1, 2, 3, 4, 5, 6, 7, 8 & 9 (A), 9, 11, 14, 16 & 19 (B) and 10, 17, 18, 20 & 21 (C). Data are represented as mean ratios of Renilla to Firefly luciferase activity (±SEM) and are normalised relative to the mock treated cells. Differences were considered statistically significant when the p value, determined according to the Student's 2 tailed paired t-test, was less than 0.05.

FIG. 12 shows the serum concentrations of HBV surface antigen detected in mice that had been subjected to the hydrodynamic injection procedure. Serum was isolated from mice on day 3 (A) and day 5 (B) then processed for detection of HBsAg using the BioRad ELISA kit. Averages were determined for each of the groups of mice and results were normalised relative to the values obtained for the mice treated with the control scrambled siRNA. Differences were considered statistically significant when the p value, determined according to the Student's 2 tailed paired t-test, was less than 0.01 (**) or 0.001 (***).

FIG. 13 shows the serum concentrations of circulating hepatitis B viral particle equivalents detected in mice that had been subjected to the hydrodynamic injection procedure. Serum was isolated from mice on day 3 (A) and day 5 (B) then processed for detection of viral DNA using a real time quantitative PCR assay. Averages of circulating viral particle equivalents (VPEs) were determined for each of the groups of mice. Differences were considered statistical significant when the p value, determined according to the Student's 2. tailed paired t-test, was less than 0.01 (**) or 0.001 (***).

FIG. 14 shows assessment of HBV Knockdown in vitro using the dual-luciferase reporter assay when cells were transfected with siRNAs containing GP modifications in the sense and antisense strands. Duplex siRNAs comprised the antisense siRNAs with indicated GP modifications that were hybridised to a sense strand with GP modification at one position (position 17, SEQ ID NO: 31) or three positions of the sense strand (positions 5, 13 & 17, SEQ ID NO: 32). Values represent the means±standard deviation of 3 replicate transfections (p<0.05 (*) or 0.01 (**)).

FIG. 15 shows assessment of HBV Knockdown in vitro using the dual-luciferase reporter assay when cells were transfected with siRNAs containing GP modifications in the sense and antisense strands. Duplex siRNAs comprised the antisense siRNAs with indicated GP modifications that were hybridised to a sense strand with three GP modifications (positions 5, 13 & 17, SEQ ID NO: 32). Values represent the means±standard deviation of 3 replicate transfections (p<0.05 (*) or 0.01 (**)).

### DETAILED DESCRIPTION OF THE INVENTION

The present invention will now be described more fully hereinafter with reference to the accompanying figures, in which some, but not all embodiments of the invention are shown.

The invention as described should not to be limited to the specific embodiments disclosed and modifications and other embodiments are intended to be included within the scope of the invention. Although specific terms are employed herein, they are used in a generic and descriptive sense only and not for purposes of limitation.

Terms used herein have their meaning recognised in the art unless otherwise indicated. According to their use here, the following terms have the meanings defined below.

As used herein the term “nucleic acid” refers to a deoxyribonucleotide or ribonucleotide polymer in either single- or double-stranded form, and unless otherwise limited, encompasses analogues of natural nucleotides that hybridise to nucleic acids in a manner similar to naturally occurring nucleotides. Unless otherwise indicated, a particular nucleic acid, sequence includes the complementary sequence thereof.

The word “nucleoside” refers to purine or pyrimidine base bound to ribose or deoxyribose sugar through a beta glycosidic link. Common examples of nucleosides are guanosine, adenosine, thymidine, cytidine and uridine.

The word “nucleotide” refers to a nucleoside that is phosphorylated on its ribose or deoxyribose moiety. The most common site of phosphorylation is at the 5′ carbon of the sugar. Nucleotide polymers form DNA or RNA. The sugar and phosphate of the polymer form the nucleic acid backbone.

“Ribose” refers to a monosaccharide found in RNA and which has the formula C₅H₁₀O₅ and “deoxyribose” refers to a monosaccharide found in DNA and which has the formula C₅H₁₀O₄.

The abbreviation “siRNA” refers to a “small interfering RNA”. siRNA's consist of a short double-stranded RNA molecule, the antisense- (guide) strand and the sense- (passenger) strand. Typically a siRNA molecule comprises a 19 bp duplex region with 3′ overhangs of 2 nucleotides. One strand is incorporated into a cytoplasmic RNA-induced silencing complex (RISC). This directs the sequence specific RNA cleavage that is effected by RISC. Mismatches between the siRNA guide and its target may cause translational suppression instead of RNA cleavage. siRNA may be synthetic or derived from processing of a precursor by Dicer.

As used herein “RNA interference” (RNAi) is the process by which synthetic siRNAs or the expression of a nucleic acid (including miR, siRNA, shRNA) causes sequence-specific degradation of complementary RNA, sequence-specific translational suppression or transcriptional gene silencing and further as used herein “RNAi-encoding sequence” refers to a nucleic acid sequence which, when expressed, causes RNA interference.

The word “Dicer” refers to an RNAse III enzyme, which digests double stranded RNA and is responsible for maturation of RNAi precursors. For example, Dicer is responsible for acting on pre-miRs to form mature miRs. “Drosha” is an RNase III enzyme that forms part of the nuclear microprocessor complex that recognises specific pri-miR secondary structures to cleave and release pre-miR sequences of approximately 60-80 nt.

The term “transcription” refers to the process of producing RNA from a DNA template. “In vitro transcription” refers to the process of transcription of a DNA sequence into RNA molecules using a laboratory medium which contains an RNA polymerase and RNA precursors and “intracellular transcription” refers to the transcription of a DNA sequence into RNA molecules, within a living cell. Further, “in vivo transcription” refers to the process of transcription of a DNA sequence into RNA molecules, within a living organism.

As used herein, the term ‘target nucleic acid’ or “nucleic acid target” refers to a nucleic acid sequence derived from a gene, in respect of which the RNAi-encoding sequence of the invention is designed to inhibit, block or prevent gene expression, enzymatic activity or interaction with other cellular or viral factors. In terms of the invention “target nucleic acid” or “nucleic acid target” encompass any nucleic acid capable of being targeted including without limitation including DNA, RNA (including pre-mRNA and mRNA or portions thereof) transcribed from DNA, and also cDNA derived from RNA.

The term “guide sequence” is equivalent to the term “antisense strand” and as used herein, refers to a short single stranded RNA fragment derived from an RNAi effecter, for example siRNA, miR, shRNA that is incorporated into RISC, and which is responsible for sequence-specific degradation or translation suppression of target RNA at a target recognition sequence. Further the term “RNAi effecter” refers to any RNA sequence (e.g. shRNA, miR and siRNA) including its precursors, which can cause RNAi.

When referring to the moieties attached to the nucleosides described herein “Guanidino group” refers to a chemical moiety that includes three nitrogen atoms and one carbon atom with the chemical structure depicted below. “Propyl group” refers to a chemical moiety that includes three carbon atoms with the chemical structure depicted below and “Guanidinopropyl group” refers to a chemical moiety that comprises a guanidino group covalently linked to a propyl component.

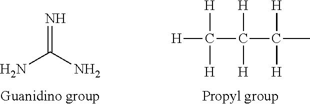

The present invention provides nucleic acid compounds which are useful in the modulation of gene expression. The nucleic acid compounds of the invention modulate gene expression by hybridising to nucleic acid target sequences. The result of the hybridisation is the loss of normal function of the target nucleic acid. In a preferred embodiment of this invention modulation of gene expression is effected via modulation of a particular RNA associated with the particular gene-derived RNA.

The invention further provides for modulation of a target nucleic acid that is a messenger RNA. The messenger RNA is degraded by the RNA interference mechanism as well as other mechanisms in which double stranded RNA/RNA structures are recognised and degraded, cleaved or otherwise rendered inoperable.

The functions of RNA to be interfered with include replication and transcription. Replication and transcription may be from an endogenous cellular template, a vector, a plasmid construct or from other sources. The functions of RNA to be interfered with may include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA.

In the context of the present invention, “modulation” and “modulation of expression” can mean either an increase (stimulation) or a decrease (inhibition) in the level or amount of a nucleic acid molecule encoding the gene, e.g., DNA or RNA. Inhibition is often the preferred form of modulation of expression and mRNA is often a preferred target nucleic acid.

The following examples are offered by way of illustration and not by way of limitation.

### Methods and Materials

All reagents were of analytical reagent grade, obtained from commercial resources and used without further purification. For synthesis, solvents with quality pro analysi were used. Dry solvents were kept over molecular sieve and column chromatography technical solvents were distilled before use.

All NMR spectra were measured on Bruker AM250 (¹H: 250 MHz, ¹³C: 63 MHz), AV300 (¹H: 300 MHz, ¹³C: 75 MHz, ³¹P: 121 MHz) and AV400 (¹H: 400 MHz, ¹³C: 101 MHz, ³¹P: 162 MHz) instruments. Chemical shifts (δ) are reported in parts per million (ppm). The following annotations were used with peak multiplicity: s, singlet; d, doublet; t, triplet; q, quartet; m, multiplet; b, broadened. J values are given in Hz. MALDI mass spectra were recorded on a Fisons VG Tofspec spectrometer and ESI mass spectra on a Fisons VG Plattform II spectrometer. High resolution mass spectra were acquired on a Thermo MALDI Orbitrap XL.

UV-Melting curves were measured on a JASCO V-650 spectrophotometer. Melting profiles of the RNA duplexes were recorded in a phosphate buffer containing NaCl (100 mM, pH 7) at oligonucleotide concentrations 2 μM for each strand at wavelength 260 nm. Each melting curve was determined in triplicate. The temperature range was 5-95° C. with a heating rate 0.5° C. The thermodynamic data were extracted from the melting curves by means of a two state model for the transition from duplex to single strands.

### Example 1

### Synthesis of the four 2′-O-guanidinopropyl-nucleoside-phosphoramidites

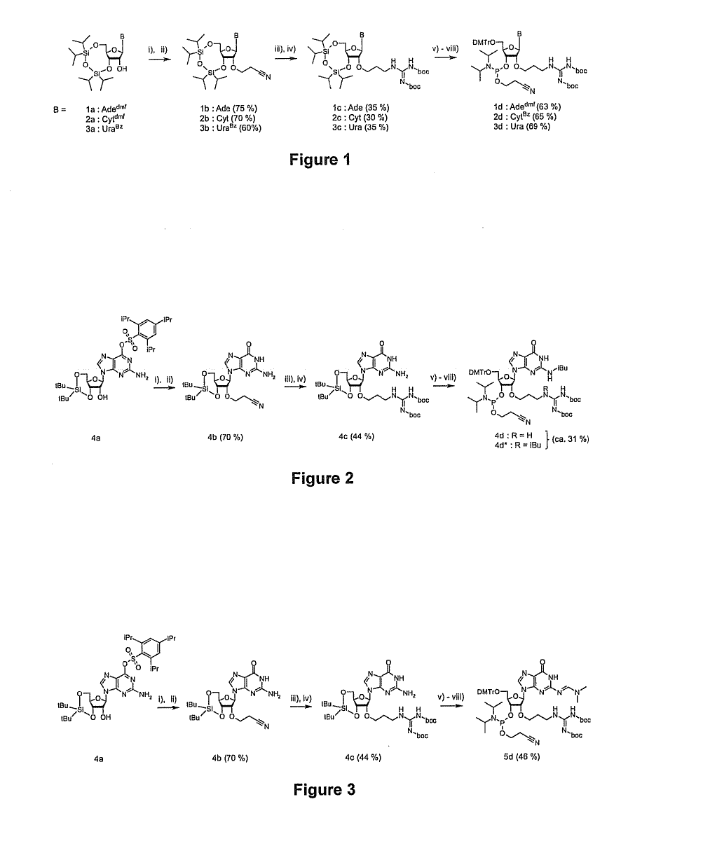

Each of the four 2′-O-guanidinopropyl-nucleoside phosphoramidites was synthesised using essentially analogous methodology. The synthesis method of the adenosine (A), cytidine (C) and uridine (U) derivatives is depicted in FIG. 1. Since a different protecting group strategy was employed to synthesise the guanosine (G) derivative, it is shown in a separate scheme (FIG. 2). Each synthesis was initiated by simultaneous protection of 5′- and the 3′-OH-groups with 1,1,3,3-tetraisopropyldisiloxane-1,3-diyl (TIPS) (for A, C and U) or di-tert-butylsilanediyl (DTBS) for guanosine. DTBS was selected for protection of G as this group has been reported to improve selectivity for the subsequent 2,4,6-triisopropylbenzenesulfonyl (TPS) protection of O⁶-position of guanosine [21]. The exocyclic amino functions of A and C were protected with dimethylaminomethylene groups employing standard conditions and a benzoyl group was attached to N³-position of U using the two phase system reported by Sekine [22]. The resulting nucleotide precursors (1a-4a) were then subjected to the first crucial step of the 2′-O-guanidinopropyl derivatisation. Employing the procedure reported by Sekine et al. [23], a Michael addition under mild conditions (CsCO₃, tert-butanol, room temperature) was performed using acrylonitrile to obtain the 2′-O-cyanoethyl derivatives. In a subsequent step the dimethylaminomethylene group of the A and C derivatives was removed with hydrazine to form the 2′-O-cyanoethyl derivatives 1b and 2b. This additional deprotection step was necessary to avoid formation of a mixture of dimethylaminomethylene protected and unprotected derivatives that result from direct application of the next reduction step. For the uridine derivative 3b, no intermediate deprotection of the N³-benzoyl group was necessary. This is because the benzoyl group was completely removed under the ammonia conditions of the following step. The O⁶-TPS group of the guanosine derivative was removed without further purification of the Michael reaction product. This was achieved after filtration and evaporation of solvents using formic acid in a mixture of dioxane and water to yield the 2′-O-cyanoethyl-guanosine derivative 4b.

In the next step, the 2′-O-cyanoethyl group was transformed into a 2′-O-aminopropyl group. Reduction with hydrogen (30 bar) with Raney-nickel as catalyst in ammonia and methanol was used to achieve this according to a procedure we previously described [24]. The hydrogenation step was sensitive to reaction conditions that included the ratio of amount of starting material to catalyst, the size of the autoclave employed and reaction time. Under optimised conditions, yields from reduction of each nucleotide derivative were. moderate (about 50%). A loss of the desired product was also confirmed by the observation that part of the amino compound was not released from the catalyst during filtration, despite being subjected to several washes with methanol. To minimise losses the crude unpurified 2′4)aminopropyl compounds were used to introduce the guanidino groups. N,N′-di-Boc-N″-triflylguanidine was employed as guanidinylation agent. The procedure we employed was initially reported by Goodman et al., in 1998 [25] and is now commercially available. Our previous studies showed that the boc groups are cleaved under the repetitive deprotection conditions during oligonucleotide synthesis when employing the TBDMS-phosphoramidite method. Also, the guanidino group undergoes no side reaction during the solid phase synthesis [26].

The guanidinylation took place with good yields (70% for 3a (A), 60% for 3b (C), around 60% for 3c (U) and approximately 90% for 4c (G)). A further advantage of the synthetic procedures described here is that it is possible to introduce diversification at the 2′-O-aminopropyl site of our compounds. With common peptide-coupling reagents, such as carbodiimides and 1-hydroxybenzotriazoles, the 2′-O-aminopropyl group can readily be modified with carboxylic acid derivatives. These include amino acids, fatty acids or carboxy-modified spermine to obtain more cationic or more lipophilic oligonucleotides [24]. Also protection of the amino group with a trifluoroacetyl group during oligonucleotide solid phase synthesis would enable postsynthetic labeling with amino-reactive fluorophore derivatives (e.g. NHS-esters or isothiocyanates) or reaction with cross linkers.

After successful guanidinylation, established reaction conditions were applied to synthesize the desired phosphoramidites (1d -4d). This entailed use of protection groups that were suitable for the TBDMS method of oligoribonucleotide synthesis. The A derivative was protected with dimethylaminomethylene at the N⁶-position, and the exocyclic amino function of the C derivative was protected with a benzoyl group. The N²-position of the G derivative was protected with an isobutyryl group. However under the reaction conditions we employed, a mixture of the desired G derivative product as well as a compound with an additional isobutyryl group on the non-boc-protected nitrogen of the guanidino group were obtained. It was difficult to separate this additional isobutyryl group using chromatography.

However, since it would be cleaved during the ammonia deprotection step at the completion of oligonucleotide synthesis, we utilised this mixture of 4d and 4d* for solid phase oligonucleotide synthesis. To synthesise U derivatives, no further protection was necessary. For synthesis of all of the 2′-O-guanidinopropyl phosphoramidites, removal of silyl protecting groups was achieved with Et₃N.3HF. The 5′-OH-group was protected with a 4,4′-dimethoxytrityl group and in a last step the 3′-OH group was converted to a phosphoramidite using 2-cyanoethyl N,N,N′,N′-tetraisopropylamino phosphane and 4,5-dicyanoimidazole as activator. Starting with the adenosine, cytidine and guanosine nucleosides, synthesis of the 2′-O-guanidinopropyl phosphoramidites took place in 10 steps and provided overall yields of 15.4% (1d), 6.3% (2d) and 7.8% (4d). Synthesis of the 2′-O-guanidinopropyl uridine phosphoramidite was performed in 8 steps with an overall yield of 11.8% (3d).

### Example 2

### Synthesis of the 2′-O-Guanidinopropyl Adenosine Phosphoramidite

3′,5′-O-(Tetraisopropyldisiloxane-1,3-diyl)-N⁶-dimethylaminomethylene adenosine (1a) was synthesised as previously described [16].

### N⁶-Dimethylaminomethylene-2′-O-cyanoethyl-3′,5′-O-(tetraisopropyldisiloxane-1,3-diyl)-adenosine (1e)

To a solution of compound 1a (3.0 g, 5.31 mmol) in tert-butanol (25 mL), freshly distilled acrylonitrile (6.7 mL, 102 mmol) and cesium carbonate (1.6 g, 4.9 mmol) were added. The mixture was stirred vigorously at room temperature for 3 h. The reaction mixture was filtered and the residue was washed with dichloromethane. The filtrate was evaporated and the residue was purified using column chromatography with ethyl acetate/methanol (99:1-95:5, v/v) to give 3.28 g (87%) of the product. ¹H NMR (400 MHz, DMSO-d₆) δ [ppm] 8.90 (s, 1H, admidine-H), 8.34 (s, 1H, H2 or H8), 8.32 (s, 1H, H2 or H8), 6.02-6.01 (m, 1H, H1′), 5.05-5.01 (m, 1H, H3′), 4.64-4.62 (m, 1H, H2′), 4.08-3.84 (m, 5H, H4′, 2×H5′, O—CH₂—CH₂—CN), 3.20 (s, 3H, N—CH₃), 3.13 (s, 3H, N—CH₃), 2.83-2.80 (m, 2H, O—CH₂—CH₂—CN), 1.10-1.00 (m, 28H, tetraisopropyl-CH and —CH₃); MS (ESI) was calculated to be 618.3 for C₂₈H₄₈N₇O₅Si₂ (M+H⁺), and found to be 618.8.

### 2′-O-Cyanoethyl-3′,5′-O-(tetraisopropyldisiloxane-1,3-diyl)-adenosine (1b)

N⁶-Dimethylaminomethylene-2′-O-cyanoethyl-3′,5′-O-(tetraisopropyldisiloxane-1,3-diyl)-adenosine (1e) (1.0 g, 1.62 mmol) was dissolved in methanol (20 mL) then hydrazine hydrate (H₂N—NH₂.H₂O; 500 μL, 10.3 mmol) was added. The reaction solution was stirred at room temperature for 3 h. The solvents were evaporated and the residue was purified using a silica gel column with ethylacetate as eluent to give 773 mg (87%) of 1b. ¹H NMR (400 MHz, DMSO-d₆) δ [ppm] 8.21 (s, 1H, H2 or H8), 8.07 (s, 1H, H2 or H8), 7.33 (bs, 2H, NH₂), 5.98-5.96 (m, 1H, H1′), 5.03-4.99 (m, 1H, H3′), 4.59-4.57 (m, 1H, H2′), 4.08-3.83 (m, 5H, H4′, 2×H5′, O—CH₂—CH₂—CN), 2.84-2.80 (m, 2H, O—CH₂—CH₂—CN), 1.09-0.97 (m, 28H, tetraisopropyl-CH and —CH₃); ¹³C NMR (101 MHz, DMSO-d₆) δ [ppm] 156.01, 152.41 (C2 or C8), 148.46, 139.26 (C2 or C8), 119.20, 118.83, 87.47 (C1′), 81.11 (C2′), 80.45 (C4′), 70.04 (C3′), 65.62 H₂—CN), 60.09 (C5′), 18.38 (O—CH₂—CH₂—CN), {17.20, 17.06, 17.05, 17.01, 16.98, 16.85, 16.81, 16.71} (tetraisopropyl-CH₃), {12.60, 12.28, 12.09, 12.04} (tetraisopropyl-CH); MS (MALDI) was calculated to be 563.8 for C₂₆H₄₃N₆O₅Si₂ (M+H⁺) and found to be 564.0.

### 2′-O-Aminopropyl-3′,5′-O-(tetraisopropyldisiloxane-1,3-diyl)-adenosine (1f)

Compound 1b (1.0 g, 1.78 mmol) was dissolved in 10 mL of methanol in a glass tube suitable for use in an autoclave. Approximately 0.5 mL of the Raney-nickel slurry was rinsed thoroughly with dry methanol and then washed into the glass tube with the solution of 1b. After addition of 5 mL methanol saturated with ammonia, the mixture was stirred for 1 h at room temperature under a hydrogen atmosphere (30 bar). The reaction mixture was filtered and the catalyst was washed several times with methanol. The filtrate was evaporated and the residue was purified using column chromatography with ethyl acetate/methanol/triethylamine (70:25:5, v/v/v) to yield 503 mg (50%) of the desired compound. When this reaction was repeated, the crude product was used for the next step without further purification. ¹H NMR (400 MHz, DMSO-d₆) δ [ppm] 8.20 (s, 1H, H2 or H8), 8.07 (s, 1H, H2 or H8), 7.32 (bs, 2H, NH₂), 5.95-5.94 (m, 1H, H1′), 4.95-4.90 (m, 1H, H3′), 4.41-4.39 (m, 1H, H2′), 4.08-3.90 (m, 3H, H4′, 2×H5′), 3.86-3.70 (m, 2H, O—CH₂—CH₂—CH₂—NH₂), 2.66-2.61 (m, 2H, O—CH₂—CH₂—CH₂—NH₂), 1.65-1.58 (m, 2H, O—CH₂—CH₂—CH₂—NH₂), 1.08-0.96 (m, 28H, tetraisopropyl-CH and —CH₃); MS (MALDI) was calculated to be 567.9 for C₂₅H₄₇N₆O₅Si₂ (M+H⁺), and found to be 567.9.

### 2′-O—(N,N′-Di-boc-guanidinopropyl)-3′,5′-O-(tetraisopropyldisiloxane-1,3-diyl)adenosine (1c)

N,N′-Di-boc-N″-triflyl guanidine (280 mg, 0.72 mmol) was dissolved in 5 mL dichloromethane then triethylamine (100 μL) was added. After cooling to 0° C., 2′-O-aminopropyl-3′,5′-O-(tetraisopropyldisiloxane-1,3-diyl)-adenosine (1f) (400 mg, 0.71 mmol) was added and the mixture was stirred for 1 h at 0° C. then for 1 h at room temperature. The reaction was diluted with dichloromethane and washed with saturated sodium bicarbonate solution and brine. The organic layer was dried over Na₂SO₄ and the solvent was evaporated. The residue was purified using column chromatography with dichloromethane/methanol (98:2, v/v) to give a yield of 402 mg (70%) of 1c. ¹H NMR (400 MHz, DMSO-d₆) δ [ppm] 11.50 (s, 1H, NH-boc), 8.45-8.41 (m, 1H, NH—CH₂—), 8.17 (s, 1H, H2 or H8), 8.06 (s, 1H, H2 or H8), 7.31 (bs, 2H, NH₂), 6.02-5.99 (m, 1H, H1′), 4.96-4.91 (m, 1H, H3′), 4.43-4.40 (m, 1H, H2′), 4.06-3.70 (m, 5H, H4′, 2×H5′, O—CH₂—CH₂—CH₂—NH—), 3.51-3.32 (m, 2H, O—CH₂—CH₂—CH₂—NH—), 1.84-1.78 (m, 2H, O—CH₂—CH₂—CH₂—NH—), 1.44 (s, 9H, C(CH₃)₃), 1.37 (s, 9H, C(CH₃)₃), 1.07-0.99 (m, 28H, tetraisopropyl-CH and —CH₃); ¹³C NMR (101 MHz, DMSO-d₆) δ [ppm] 163.00, 156.00, 155.07, 152.40 (C2 or C8), 151.96, 148.48, 139.04 (C2 or C8), 119.21, 87.69 (C1′), 82.70, 81.26 (C2′), 80.41 (C4′), 77.87, 69.99 (C3′), 69.63 (O—CH₂—CH₂—CH₂—NH—), 60.13 (C5′), 38.41 (O—CH₂—CH₂—CH₂—NH—), 28.71 (O—CH₂—CH₂—CH₂—NH—), 27.85 (C(CH₃)₃), 27.44 (C(CH₃)₃), {17.19, 17.05, 17.03, 17.00, 16.95, 16.82, 16.74, 16.68} (tetraisopropyl-CH₃), {12.59, 12.28, 12.09, 12.01} (tetraisopropyl-CH); MS (MALDI) was calculated to be 810.1 for O₃₆H₆₅N₈O₉Si₂ (M+H⁺), and found to be 808.3.

### N⁶-Dimethylaminomethylene-2′-O—(N,N′-di-boc-guanidinopropyl)-3′,5′-O-(tetraisopropyldisiloxane-1,3-diyl)-adenosine (1g)

Compound 1c (500 mg, 0.61 mmol) was dissolved in methanol (5 mL) and N,N-dimethylformamide dimethyl acetale (500 μL, 3.7 mmol) was added. The reaction was stirred at room temperature overnight and the solvents were evaporated. The crude product was used for further reactions without purification.

### N⁶-Dimethylaminomethylene-2′-O—(N,N′-di-boc-guanidinopropyl)-adenosine (1h)

N⁶-Dimethylaminomethylene-2′-O—(N,N′-di-boc-guanidinopropyl)-3′,5′-O-(tetraisopropyldisiloxane-1,3-diyl)-adenosine (1g) (500 mg, 0.58 mmol) was dissolved in tetrahydrofurane (5 mL) and triethylammonium trihydrofluoride (Et₃N.3HF; 330 μL, 2.0 mmol) was added. The mixture was stirred at room temperature for 1.5 h, then the solvent was evaporated. The residue was purified by column chromatography with ethyl acetate/methanol (98:2-9:1, v/v) giving 300 mg (83%) of the desired product. ¹H NMR (400 MHz, DMSO-d₆) δ [ppm] 11.47 (s, 1H, NH-boc), 8.92 (s, 1H, N⁶═CH—NMe₂), 8.50 (s, 1H, H2 or H8), 8.41 (s, 1H, H2 or H8), 8.33-8.29 (m, 1H, NH—CH₂—), 6.11-6.09 (m, 1H, H1′), 5.20-5.24 (m, 1H, 5′-OH), 5.18-5.16 (m, 1H, 3′-OH), 4.46-4.43 (m, 1H, H2′), 4.36-4.32 (m, 1H, H3′), 4.01-3.98 (m, 1H, H4′), 3.72-3.46 (4H, 2×H5′, O—CH₂—CH₂—CH₂—NH—), 3.33-3.28 (m, 2H, O—CH₂—CH₂—CH₂—NH—), 3.20 (s, 3H, N—CH₃), 3.13 (s, 3H, N—CH₃), 1.74-1.68 (m, 2H, O—CH₂—CH₂—CH₂—NH—), 1.45 (s, 9H, C(CH₃)₃), 1.37 (s, 9H, C(CH₃)₃); ¹³C NMR (101 MHz, DMSO-d₆) δ [ppm] 162.97, 159.22, 157.97 (N⁶═CH—NMe₂), 155.09, 151.89, 151.77 (C2 or C8), 151.00, 141.08 (C2 or C8), 125.70, 85.91 (C1′), 85.74 (C4′), 82.72, 81.02 (C2′), 77.99, 68.72 (C3′), 67.88 (O—CH₂—CH₂—CH₂—NH—), 61.04 (C5′), 40.56 (N—CH₃), 37.86 (O—CH₂—CH₂—CH₂—NH—), 34.45 (N—CH₃), 28.57 (O—CH₂—CH₂—CH₂—NH—), 27.87 (C(CH₃)₃), 27.51 (C(CH₃)₃); MS (MALDI) was calculated to be 622.7 for C₂₇H₄₄N₉O₈ (M+H⁺), and found to be 624.6.

### N⁶-Dimethylaminomethylene-2′-O—(N,N′-di-boc-guanidinopropyl)-5′-O-(4,4′-dimethoxytrityl)-adenosine (1i)

N⁶-Dimethylaminomethylene-2′-O—(N,N′-di-boc-guanidinopropyl)-adenosine (1h) (1.0 g, 1.6 mmol) was dissolved in dry pyridine (20 mL). 4,4′-Dimethoxytrityl chloride (660 mg, 1.95 mmol) was added and the reaction was stirred at room temperature overnight. The solution was diluted with dichloromethane and washed with saturated sodium bicarbonate solution. After evaporation of the solvents the residue was purified on a silica gel column with dichloromethane/methanol (98:2, v/v) containing 0.5% triethylamine, and 1.32 g (90%) of the tritylated compound was obtained. ¹H NMR (400 MHz, DMSO-d₆) δ [ppm] 11.48 (s, 1H, NH-boc), 8.90 (s, 1H, N⁶═CH—NMe₂), 8.38-8.34 (m, 3H, H2, H3, NH—CH₂—), 7.37-7.34 (m, 2H, DMTr), 7.27-7.17 (m, 7H, DMTr), 6.84-6.79 (m, 4H, DMTr), 6.14-6.13 (m, 1H, H1′), 5.18-5.15 (m, 1H, 3′-OH), 4.57-4.54 (m, 1H, H2′), 4.47-4.42 (m, 1H, H3′), 4.14-4.08 (m, 1H, H4′), 3.72-3.71 (m, 6H, 2×OCH₃), 3.70-3.56 (m, 2H, O—CH₂—CH₂—CH₂—NH—), 3.37-3.32 (m, 2H, O—CH₂—CH₂—CH₂—NH—), 3.24-3.21 (m, 2H, 2×H5′), 3.19 (s, 3H, N—CH₃), 3.12 (s, 3H, N—CH₃), 1.77-1.70 (m, 2H, O—CH₂—CH₂—CH₂—NH—), 1.44 (s, 9H, C(CH₃)₃), 1.35 (s, 9H, C(CH₃)₃); ¹³C NMR (101 MHz, DMSO-d₆) δ [ppm] 162.98, 159.15, 157.97, 157.94, 157.91, 157.85 (N⁶═CH—NMe₂), 155.09, 151.88 (C2 or C8), 151.06, 144.73, 141.18 (C2 or C8), 135.44, 135.37, {129.60, 129.56, 127.64, 127.59, 126.53} (DMTr), 125.70, 112.99 (DMTr), 86.14 (C1′), 85.34, 82.97 (C4′), 82.70, 80.36 (C2′), 77.96, 69.08 (C3′), 68.21 (O—CH₂—CH₂—CH₂—NH—), 63.40 (C5′), 54.88 (OCH₃), 40.54 (N—CH₃), 37.97 (O—CH, —CH₂—CH₂—NH—), 34.44 (N—CH₃), 28.56 (O—CH₂—CH₂—CH₂—NH—), 27.84 (C(CH₃)₃), 27.50 (C(CH₃)₃); MS (MALDI) was calculated to be 925.1 for C₄₈H₆₂N₆O₁₀ (M+H⁺), and found to be 924.9.

### N⁶-Dimethylaminomethylene-2′-O—(N,N′-di-boc-guanidinopropyl)-5′-O-(4,4′-dimethoxytrityl)-adenosine 3′-(cyanoethyl)-N,N-diisopropyl phosphoramidite (1d)

N⁶-Dimethylaminomethylene-2′-O—(N,N′-di-boc-guanidinopropyl)-5′-O-(4,4′-dimethoxytrityl)-adenosine (1i) (320 mg, 346 μmol) was dissolved in dichloromethane (8 mL). 2-cyanoethyl N,N,N′,N′-tetraisopropylamino phosphane (132 μL, 415 μmol) and 4,5-dicyanoimidazole (47 mg, 398 μmol) were added. The mixture was stirred at room temperature. After 3 h, TLC revealed that some starting material did not react. An additional 0.6 equivalents of the phosphitylating agent as well as the catalyst were therefore added. After 4 h the reaction was complete. The mixture was diluted with dichloromethane, washed with saturated sodium bicarbonate solution and the organic layer was dried over MgSO₄. The solvent was evaporated and the residue dissolved in a small amount of dichloromethane (ca. 5 mL). This solution was added dropwise into a flask with hexane (500 ml) to form a white precipitate. Two thirds of the solvent were evaporated and the remaining solvent was decanted from the solid. The precipitated product was redissolved in benzene and lyophilised to give 329 mg (84%) of *Id as a white powder. ¹H NMR (300 MHz, acetone-d₆) δ [ppm] 11.65 (s, 1H, NH-boc) 8.95-8.93 (m, 1H, N⁶═CH—NMe₂), 8.42-8.27 (m, 3H, H2, H3, NH—CH₂—), 7.50-7.46 (m, 2H, DMTr), 7.38-7.17 (m, 7H, DMTr), 6.87-6.80 (m, 4H, DMTr), 6.28-6.26 (m, 1H, H1′), 4.96-4.79 (m, 2H, H2′, H3′) 4.45-4.37 (m, 1H, H4′), 4.05-3.35 (m, 16H), 3.25 (s, 3H, N—CH₃), 3.18 (s, 3H, N—CH₃), 2.85 (m, 1H, cyanoethyl), 2.64-2.60 (m, 1H, cyanoethyl), 1.90-1.82 (m, 2H, O—CH₂—CH₂—CH₂—NH—), 1.50-1.49 (m, 9H, C(CH₃)₃), 1.42-1.40 (m, 9H, C(CH₃)₃), 1.25-1.10 (m, 12H, iPr-CH₃); ³¹P NMR (121 MHz, acetone-d₆) δ [ppm] 149.6, 149.3; MS (ESI) was calculated to be 1125.3 for C₆₇H₇₉N₁₁O₁₁P (M+H⁺), and found to be 1125.7.

### Example 3

### Synthesis of the 2′-O-Guanidinopropyl Cytidine Phosphoramidite

N⁴-Dimethylaminomethylene-3′,5′-O-(tetraisopropyldisiloxane-1,3-diyl)-cytidine (2a) was synthesised according to a previously described procedure [29].

### N⁴-Dimethylaminomethylene-2′-O-cyanoethyl-3′,5′-O-(tetraisopropyldisiloxane-1,3-diyl)-cytidine (2e)

Compound 2a (4 g, 7.39 mmol) was dissolved in acrylonitrile (8 mL, 122 mmol) and tert-Butanol (35 mL). Cesium carbonate (1.8 g, 5.52 mmol) was added and the reaction was stirred for 2.5 h at room temperature. The mixture was filtered over celite, the solvents evaporated, and then the residue was purified using column chromatography. Ethyl acetate was initially used as solvent then changed to ethyl acetate/methanol (9:1, v/v) after the unpolar impurities had passed through the column. A yield of 3.78 g (86%) of the product were obtained. ¹H NMR (400 MHz, DMSO-d₆) δ [ppm] 8.62 (s, 1H, N⁴═CH—NMe₂), 7.88 (d, 1H, J=7.3 Hz, H6), 5.90 (d, 1H, J=7.3 Hz, H5), 5.65 (s, 1H, H1′), 4.22-3.91 (m, 7H), 3.17 (s, 3H, N—CH₃), 3.04 (s, 3H, N—CH₃), 2.86-2.82 (m, 2H, O—CH₂—CH₂—CN), 1.07-0.96 (m, 28H, tetraisopropyl-CH and —CH₃); ¹³C NMR (101 MHz, DMSO-d₆) δ [ppm] 171.21 (C4), 157.77 (N⁴═CH—NMe₂), 154.57 (C2), 140.61 (C6), 118.86 (O—CH₂—CH₂—CN), 101.14 (C5), 88.99 (C1′), 81.42, 80.69, 67.83, 65.22 (O—CH₂—CH₂—CN), 59.39 (C5′), 40.79 (N—CH₃), 34.71 (N—CH₃), 18.18 (O—CH₂—CH₂—CN), {17.22, 17.11, 17.04, 16.97, 16.84, 16.72, 16.69, 16.61} (tetraisopropyl-CH₃), {12.60, 12.20, 11.88} (tetraisopropyl-CH); MS (ESI) was calculated to be 594.9 for C₂₇H₄₆N₆O₆Si₂ (M+H⁺) and found to be 594.9.

### 2′-O-Cyanoethyl-3′,5′-O-(tetraisopropyldisiloxane-1,3-diyl)-cytidine (2b)

N⁴-Dimethylaminomethylene-2′-O-cyanoethyl-3′,5′-O-(tetraisopropyldisiloxane-1,3-diyl)-cytidine (2e) (1.0 g, 1.68 mmol) was dissolved in methanol (10 mL) and hydrazine hydrate (500 μL, 10.3 mmol) was added. The mixture was stirred for 1 h at room temperature and then the solvents were evaporated. The residue was purified on a silica gel column with ethyl acetate/methanol (95:5, v/v) to give 745 mg (82%) of 2b. ¹H NMR (400 MHz, DMSO-d₆) δ [ppm] 7.69 (d, 1H, J=7.4 Hz, H6), 7.21 (s, 2H, NH₂), 5.69 (d, 1H, J=7.4 Hz, H5), 5.61 (s, 1H, H1′); 4.19-3.90 (m, 7H), 2.90-2.76 (m, 2H, O—CH₂—CH₂—CN), 1.07-0.97 (m, 28H, tetraisopropyl-CH and —CH₃); ¹³C NMR (101 MHz, DMSO-d₆) δ [ppm] 165.70, 154.60, 139.36 (C6), 118.89, 93.30 (C5), 88.66 (C1′), 81.55 (C2′), 80.49, 67.92, 65.19 (O—CH₂—CH₂—CN), 59.44 (C5′), 18.20 (O—CH₂—CH₂—CN), {17.23, 17.11, 17.05, 16.98, 16.85, 16.73, 16.72, 16.63} (tetraisopropyl-CH₃), {12.62, 12.28, 12.21, 11.88} (tetraisopropyl-CH); MS (ESI) was calculated to be 539.8 for C₂₄H₄₃N₄O₆Si₂ (M+H⁺) and found to be 540.0.

### 2′-O-Aminopropyl-3′,5′-O-(tetraisopropyldisiloxane-1,3-diyl)-cytidine (2f)

Compound 2b (500 mg, 928 μmol) was dissolved in 10 mL of methanol in a glass tube. Approximately 0.5 mL of the Raney-nickel sediment was washed thoroughly with dry methanol and was rinsed into the glass tube with the solution of 2b. After addition of 5 mL methanol saturated with ammonia, the mixture was stirred for 1 h at room temperature under a hydrogen atmosphere (30 bar). The reaction mixture was filtered through celite and the catalyst was washed several times with methanol. The solvent was evaporated and the residue was purified on a silica gel column using ethyl acetate/methanol/triethylamine (60:35:5) to give 251 mg (50%) of the product. When this procedure was repeated, the crude material after filtration and evaporation was used in further reactions without purification. ¹H NMR (400 MHz, DMSO-d₆) δ [ppm] 7.69 (d, 1H, J=7.2 Hz, H6), 7.18 (bs, 2H, ar. NH₂), 5.68 (d, 1H, J=7.5 Hz, H5), 5.60 (s, 1H, H1′), 4.18-3.76 (m, 7H), 2.70-2.66 (m, 2H, O—CH₂—CH₂—CH₂—NH₂), 1.68-1.61 (m, 2H, O—CH₂—CH₂—CH₂—NH₂), 1.07-0.95 (m, 28H, tetraisopropyl-CH and —CH₃); MS (MALDI) was calculated to be 643.8 for C₂₄H₄₇N₄O₆Si₂ (M+H⁺) and found to be 544.6.

### 2′-O—(N,N′-Di-boc-guanidinopropyl)-3′,5′-O-(tetraisopropyldisiloxane-1,3-diyl)-cytidine (2c)

N,N′-Di-boc-N″-triflyl guanidine (360 mg, 920 μmol) was dissolved in 5 mL dichloromethane and triethylamine (125 μL) then added. After cooling to 0° C., 2′-O-Aminopropyl-3′,5′-O-(tetraisopropyldisiloxane-1,3-diyl)-cytidine (2f) (500 mg, 922 μmol) was added and the solution was stirred for 1 h at 0° C. and then 1 h at room temperature. The reaction was diluted with dichloromethane and washed with saturated sodium bicarbonate solution and brine. The combined organic layers were dried over Na₂SO₄ and after evaporating the solvent the residue was purified using column chromatography with dichloromethane/methanol (98:2-95:5, v/v) to give 434 mg (60%) of 2c. ¹H NMR (400 MHz, DMSO-d₆) δ [ppm] 11.48 (s, 1H, NH-boc), 8.38-8.35 (m, 1H, NH—CH₂—), 7.67 (d, 1H, J=7.4 Hz, H6), 7.19 (bs, 2H, NH₂), 5.68 (d, 1H, J=7.4 Hz, H5), 5.63 (s, 1H, H1′), 4.17-3.78 (m, 7H), 3.49-3.33 (m, 2H, O—CH₂—CH₂—CH₂—NH—), 1.84-1.77 (m, 2H, O—CH₂—CH₂—CH₂—NH—), 1.45 (m, 9H, C(CH₃)₃), 1.38 (m, 9H, C(CH₃)₃), 1.06-0.96 (m, 28H, tetraisopropyl-CH and —CH₃); ¹³C NMR (101 MHz, DMSO-d₆) δ [ppm] 165.61, 162.99, 155.04, 154.52, 151.94, 139.45 (C6), 93.21 (C5), 88.97 (C1′), 82.66, 81.76 (C2′), 80.36 (C4′), 77.86, 69.11 (O—CH₂—CH₂—CH₂—NH—), 68.27 (C3′), 59.51 (C5′), 38.28 (O—CH₂—CH₂—CH₂—NH—), 28.61 (O—CH₂—CH₂—CH₂—NH—), 27.86 (C(CH₃)₃), 27.44 (C(CH₃)₃), {17.22, 17.10, 17.03, 16.96, 16.83, 16.70, 16.68, 16.60} (tetraisopropyl-CH₃), {12.59, 12.26, 12.21, 11.87} (tetraisopropyl-CH); MS (MALDI) was calculated to be 786.1 for C₃₆H₆₅N₆O₁₀Si₂ (M+H⁺) and found to be 786.4.

### N⁴-Benzoyl-2′-O—(N,N′-di-boc-guanidinopropyl)-3′,5′-O-(tetraisopropyldisiloxane-1,3-diyl)-cytidine (2g)

Compound 2c (1.0 g, 1.27 mmol) was dissolved in dry pyridine (10 mL) and the solution was cooled in an ice bath. Benzoyl chloride (240 μL, 2.06 mmol) was added and the reaction solution was stirred at 0° C. for 1 h. The reaction was quenched with water and ammonia (25% in water; 3 mL) was added. The mixture was then stirred for 30 minutes at room temperature. The solvents were evaporated and the residue was dissolved in dichloromethane and washed with saturated sodium bicarbonate solution. The organic layer was dried over Na₂SO₄ and after evaporating the solvent, the residue was purified by column chromatography using dichloromethane/methanol (98:2, v/v) and 950 mg (84%) of the product were obtained. ¹H NMR (400 MHz, DMSO-d₆) δ [ppm] 11.50 (s, 1H, NH), 11.31 (s, 1H, NH), 8.40-8.37 (m, 1H, NH—CH₂—), 8.15 (d, 1H, J=7.3 Hz, H6), 8.03-7.99 (m, 2H, benzoyl), 7.65-7.60 (m, 1H, benzoyl), 7.53-7.49 (m, 2H, benzoyl), 7.38 (d, 1H, J=7.3 Hz, H5), 5.73 (s, 1H, H1′), 4.24-4.13 (m, 3H, H3′, H4′, H5′), 4.02-4.03 (m, 1H, H2′), 3.97-3.92 (m, 1H, H5′), 3.87-3.83 (m, 2H, O—CH₂—CH₂—CH₂—NH—), 3.52-3.35 (m, 2H, O—CH₂—CH₂—CH₂—NH—), 1.87-1.80 (m, 2H, O—CH₂—CH₂—CH₂—NH—), 1.45 (m, 9H, C(CH₃)₃), 1.38 (m, 9H, C(CH₃)₃), 1.08-0.95 (m, 28H, tetraisopropyl-CH and —CH₃); ¹³C NMR (101 MHz, DMSO-d₆) J[ppm] 167.21, 163.14, 163.00, 155.06, 154.01, 151.97, 143.37 (C6), 132.97, 132.64, 128.31, 95.61 (C5), 89.46 (C1′), 82.67, 81.30 (C2′), 80.86 (C4′), 77.86, 69.26 (O—CH₂—CH₂—CH₂—NH—), 67.95 (C3′), 59.38 (C5′), 38.28 (O—CH₂—CH₂—CH₂—NH—), 28.62 (O—CH₂—CH₂—CH₂—NH—), 27.86 (C(CH₃)₃), 27.43 (C(CH₃)₃), {17.22, 17.11, 17.04, 16.97, 16.89, 16.73, 16.71, 16.66} (tetraisopropyl-CH₃), {12.56, 12.29, 12.22, 11.86} (tetraisopropyl-CH); MS (ESI) was calculated to be 890.2 for C₄₂H₆₉N₆O₁₁Si₂ (M+H⁺), and found to be 890.4.

### N⁴-Benzoyl-2′-O—(N,N′-di-boc-guanidinopropyl)-cytidine (2h)

N⁴-Benzoyl-2′-O—(N,N′-di-boc-guanidinopropyl)-3′,5′-O-(tetraisopropyldisiloxane-1,3-diyl)-cytidine (2g) (900 mg, 1.01 mmol) was dissolved in tetrahydrofurane (20 mL). Triethylamine trihydrofluoride (Et₃N.3HF; 560 μL, 3.54 mmol) was added and the solution was stirred at room temperature for 2 h. The solvent was evaporated and the residue was purified using column chromatography with dichloromethane/methanol (98:2-97:3, v/v) to give 607 mg (93%) of the product as a pale yellow foam. ¹H NMR (300 MHz, DMSO-d₆) δ [ppm] 11.50 (s, 1H, NH), 11.28 (bs, 1H, NH), 8.57 (d, 1H, J=7.5 Hz, H6), 8.40-8.35 (m, 1H, NH—CH₂—), 8.02-7.98 (m, 2H, benzoyl), 7.66-7.60 (m, 1H, benzoyl), 7.54-7.48 (m, 2H, benzoyl), 7.34 (d, 1H, J=7.2 Hz, H5), 5.86-5.85 (m, 1H, H1′), 5.24 (t, 1H, J=5.0 Hz, 5′-OH), 4.98 (d, 1H, J=6.8 Hz, 3′-OH), 4.12-3.60 (m, 7H), 3.44-3.37 (m, 2H, O—CH₂—CH₂—CH₂—NH—), 1.85-1.76 (m, 2H, O—CH₂—CH₂—CH₂—NH—), 1.46 (m, 9H, (C(CH₃)₃), 1.38 (m, 9H, C(CH₃)₃); ¹³C NMR (75 MHz, acetone-d₆) δ [ppm] 169.22, 165.59, 164.82, 157.83, 156.15, 154.80, 147.10 (C6), 135.58, 134.60, 130.46, 130.05, 97.67 (C5), 91.39 (C1′), 86.19 (C4′), 84.69 (C(CH₃)₃), 84.62 (C2′), 79.88 (C(CH₃)₃), 70.71 (O—CH₂—CH₂—CH₂—NH—), 69.63 (C3′), 61.46 (C5′), 40.29 (O—CH₂—CH₂—CH₂—NH—), 30.86 (O—CH₂—CH₂—CH₂—NH—), 29.46 (C(CH₃)₃), 29.14 (C(CH₃)₃); HRMS (MALDI) was calculated to be 647.3035 for C₃₀H₄₃N₆O₁₀ (M+H⁺), and found to be 647.3031.

### N⁴-Benzoyl-2′-O—(N,N′-di-boc-guanidinopropyl)-5′-O-(4,4′-dimethoxytrityl)-cytidine (2i)

N⁴-Benzoyl-2′-O—(N,N′-di-boc-guanidinopropyl)-cytidine (2h) (516 mg, 798 μmol) was dissolved in dry pyridine (20 mL) and the solution was cooled in an ice bath. 4,4′-Dimethoxytrityl chloride (515 mg, 1.52 mmol) was added and the mixture was stirred overnight while the bath came up to room temperature. The reaction was quenched with methanol (10 mL) and the solvents were evaporated. The residue was purified by column chromatography using dichloromethane/methanol (99:1-98:2, v/v). The column was packed with solvent containing 1% triethylamine to yield 715 mg (94%) of the product as a pale yellow foam. ¹H NMR (400 MHz, DMSO-d₆) δ [ppm] 11.50 (s, 1H, NH), 11.29 (bs, 1H, NH), 8.43-8.37 (m, 2H, H6, NH—CH₂—), 8.02-7.99 (m, 2H, benzoyl), 7.65-7.60 (m, 1H, benzoyl), 7.54-7.50 (m, 2H, benzoyl), 7.43-7.25 (m, 9H, DMTr), 7.18-7.15 (m, 1H, H5), 6.94-6.91 (m, 4H, DMTr), 5.88 (s, 1H, H1′), 5.04 (d, 1H, J=7.3 Hz, 3′-OH), 4.34-4.28 (m, 1H, H3′), 4.13-4.10 (m, 1H, H4′), 3.94-3.87 (m, 2H, H2′, 1×O—CH₂—CH₂—CH₂—NH—), 3.76 (s, 6H, 2×OCH₃), 3.76-3.70 (m, 1H, 1×O—CH₂—CH₂—CH₂—NH—), 3.46-3.36 (m, 4H, 2×H5′, O—CH₂—CH₂—CH₂—NH—), 1.86-1.80 (m, 2H, O—CH₂—CH₂—CH₂—NH—), 1.42 (m, 9H, C(CH₃)₃), 1.36 (m, 9H, C(CH₃)₃); ¹³C NMR (75 MHz, DMSO-d₆) δ [ppm] 167.19, 163.02, 158.11, 158.08, 155.12, 154.07, 151.93, 144.24 (C6), 135.47, 135.11, 133.06, 132.62, 129.70, 129.55, 128.35, 127.85, 127.73, 126.78, 113.19, 95.93 (C5), 88.99 (C1′), 85.90, 82.67 (C(CH₃)₃), 81.93 (C2′), 81.44 (C4′), 77.94 (C(CH₃)₃), 68.44 (O—CH₂—CH₂—CH₂—NH—), 67.62 (C3′), 61.36 (C5′), 54.91 (OCH₃), 54.90 (OCH₃), 38.17 (O—CH₂—CH₂—CH₂—NH—), 28.59 (O—CH₂—CH₂—CH₂—NH—), 27.87 (C(CH₃)₃), 27.48 (C(CH₃)₃); HRMS (MALDI) was calculated to be 971.4161 for C₅₁H₆₀N₆O₁₂Na (M+Na⁺), and found to be 971.4181.

### N⁴-Benzoyl-2′-O—(N,N′-di-boc-guanidinopropyl)-5′-O-(4,4′-dimethoxytrityl)-cytidine 3′-(cyanoethyl)-N,N-diisopropyl phosphoramidite (2d)

N⁴-Benzoyl-2′-O—(N,N′-di-boc-guanidinopropyl)-5′-O-(4,4′-dimethoxytrityl)-cytidine (2i) (683 mg, 720 μmol) was dissolved in dichloromethane (15 mL). 2-cyanoethyl N,N,N′,N′-tetraisopropylamino phosphane (274 μL, 864 μmol) and 4,5-dicyanoimidazole (98 mg, 828 μmol) were added. After stirring at room temperature for 5 h, TLC revealed that some starting material had not reacted. Therefore 10 mg of 4,5-dicyanoimidazole and 30 μL of the phosphitylation agent were added and the reaction was stirred at room temperature overnight. The solution was diluted with dichloromethane and washed with saturated sodium bicarbonate solution. After drying the organic layer over MgSO₄ the solvent was evaporated and the residue was dissolved in a small amount (5 mL) of dichloromethane. This solution was dripped into a flask with hexane (500 mL) to form a white precipitate. Two thirds of the solvent was evaporated and the residual solvent was decanted carefully. The precipitate was redissolved in benzene and lyophilised to give 738 mg (89%) of 2d. According to ³¹P NMR spectrum the product was still containing a small amount of the hydrolysed phosphitylation reagent but this did not interfere with the oligonucleotide synthesis. ¹H NMR (400 MHz, DMSO-d₆) δ [ppm] 11.50-11.48 (m, 1H, NH), 11.25 (bs, 1H, NH), 8.52-8.45 (m, 1H, H6), 8.39-8.34 (m, 1H, NH—CH₂—), 8.01-7.98 (m, 2H, benzoyl), 7.66-7.61 (m, 1H, benzoyl), 7.53-7.49 (m, 2H, benzoyl), 7.45-7.25 (m, 9H, DMTr), 7.13-7.09 (m, 1H, H5), 6.93-6.89 (m, 4H, DMTr), 5.95-5.92 (m, 1H, H1′), 4.56-4.38 (m, 1H, H3′), 4.31-4.28 (m, 1H, H4′), 4.07-3.29 (m, 17H), 2.90-2.57 (m, 2H, cyanoethyl), 1.86-1.78 (m, 2H, O—CH₂—CH₂—NH—), 1.40-1.35 (m, 18H, 2×C(CH₃)₃), 1.20-0.93 (m, 12H, iPr-CH₃); ³¹P NMR (162 MHz, DMSO-d₆) δ [ppm] 148.4, 148.0 (The signal of the hydrolised phosphitylation reagent appears at 13.9 ppm); HRMS (MALDI) was calculated to be 1149.5421 for C₆₀H₇₈N₈O₁₃P (M+H⁺), was found to be 1149.5447.

### Example 4

### Synthesis of the 2′-O-Guanidinopropyl Uridine Phosphoramidite

N³-Benzoyl-3′,5′-O-(tetraisopropyldisiloxane-1,3-diyl)-uridine (3a) was synthesised according to a previously described procedure [22].

### N³-Benzoyl-2′-O-cyanoethyl-3′,5′-O-(tetraisopropyldisiloxane-1,3-diyl)-uridine (3b)

Compound 3a (1.14 g, 1.93 mmol) was dissolved in 9.6 mL of tert-butanol. Freshly distilled acrylonitrile (2.5 mL, 38.6 mmol) was added. After addition of cesium carbonate (645 mg, 1.98 mmol) the reaction was stirred for 4 h at room temperature. The reaction solution was filtered over celite. The residue was washed with 100 mL of dichloromethane. The filtrate was evaporated in vacuum. Purification via column chromatography in dichloromethane/ethyl acetate (99:1-95:5, v/v) yielded 746 mg (60%) of the desired product as a white powder. ¹H NMR (250 MHz, acetone-d₆) δ [ppm] 8.03-7.99 (m, 3H, H6, benzoyl), 7.79-7.72 (m, 1H, benzoyl), 7.61-7.55 (m, 2H, benzoyl), 5.80-5.74 (m, 2H, H5, H1′), 4.50-3.94 (m, 7H), 2.80-2.75 (m, 2H, O—CH₂—CH₂—CN), 1.17-1.07 (m, 28H, tetraisopropyl-CH and —CH₃); ¹³C NMR (63 MHz, acetone-d₆) δ [ppm] 171.11, 163.78, 151.06, 141.48, 136.94, 133.92, 132.20, 131.13, 119.80, 102.75, 91.47, 84.05, 83.69, 70.65, 68.08, 61.60, 20.35, 18.92, 18.91, 18.75, 18.73, 18.64, 18.50, 18.46, 18.40, 15.23, 14.79, 14.72, 14.43; HRMS was calculated to be 666.2637 for C₃₁H₄₅N₃O₈Si₂Na (M+Na⁺) and found to be 666.2647.

### 2′-O-(Aminopropyl)-3′,5′-O-(tetraisopropyldisiloxane-1,3-diyl)-uridine (3e)

Compound 3b (500 mg, 0.78 mmol) was dissolved in 10 mL of methanol in a glass tube suitable for the applied autoclave. Approximately 0.5 mL of the Raney-nickel slurry was put on a glass filter, washed thoroughly with dry methanol and rinsed into the glass tube with the solution of 3b. After addition of 5 mL methanol saturated with ammonia, the mixture was stirred for 1 h at room temperature in an autoclave under a hydrogen atmosphere (30 bar). The reaction solution was decanted from the catalyst into a glass filter. The catalyst was washed several times with methanol and the solvent was removed from the combined filtrates under reduced pressure. The product was purified on a silica gel column initially using dichloromethane/ethyl acetate (7:3-0:1, v/v) and thereafter ethyl acetate/methanol/triethylamine (6:3.5:0.5, v/v/v) to obtain 253 mg (60%) of a white powder. When we repeated the reduction we used the crude product after filtration and evaporation for further derivatisation. ¹H NMR (250 MHz, acetone-d₆) δ [ppm] 7.81 (d, 1H, J=8.1 Hz, H6), 5.71 (s, 1H, H1′), 5.53 (d, 1H, J=8.1 Hz, H5), 4.39-4.34 (m, 1H, H3′), 4.28-4.23 (m, 1H, H5′), 4.14-4.03 (m, 3H, H2′, H4′, H5′), 3.97-3.81 (m, 2H, O—CH₂—CH₂—CH₂—NH₂), 3.37-3.25 (m, 2H, O—CH₂—CH₂—CH₂—NH), 1.92-1.82 (m, 2H, O—CH₂—CH₂—CH₂—NH₂), 1.14-1.05 (m, 28H, tetraisopropyl-CH and —CH₃); ¹³C NMR (63 MHz, acetone-d₆) δ [ppm] 167.18, 164.59, 151.93, 141.033, 102.82, 91.15, 83.77, 83.50, 70.88, 70.85, 61.78, 49.36, 33.03, 18.91, 18.90, 18.74, 18.61, 18.49, 18.47, 18.40, 15.19, 14.82, 14.71, 14.41; HRMS (MALDI) was calculated to be 544.2869 for C₂₄H₄₆N₃O₇Si₂ (M+H⁺), and found to be 544.2880.

### 2′-O—(N,N′-Di-boc-guanidinopropyl)-3′,5′-O-(tetraisopropyldisiloxane-1,3-diyl)-uridine (3c)

N,N′-Di-boc-N″-triflyl guanidine (320 mg, 0.82 mmol) was dissolved in 3.6 mL dichloromethane and triethylamine (150 μL) was added. The solution was cooled in an ice bath and 2′-O-(Aminopropyl)-3′,5′-O-(tetraisopropyldisiloxane-1,3-diyl)-uridine (3e) (490 mg, 0.9 mmol) was added. After 15 min the reaction mixture was removed from the ice bath was and stirred for 2.5 h at room temperature. The reaction solution was washed with saturated sodium bicarbonate solution and brine. After drying over Na₂SO₄ the solvent was evaporated in vacuum. The crude product was purified using column chromatography with dichloromethane/methanol (96:4-94:6, v/v). 410 mg (58%) of compound 3c were obtained. ¹H NMR (400 MHz, DMSO-d₆) δ [ppm] 11.49 (s, 1H, NH), 11.37 (m, 1H, NH_{uridine}), 8.40-8.37 (m, 1H, NH—CH₂—), 7.64 (d, 1H, J=7.9 Hz, H6), 5.64 (s, 1H, H1′), 5.53 (d, 1H, J=7.9 Hz, H5), 4.25-4.22 (H3′), 4.13-4.09 (m, 1H, H5′), 4.06-4.05 (m, 1H, H2′), 4.03-4.00 (m, 1H, H4′), 3.93-3.89 (m, 1H, H5′), 3.84-3.70 (m, 2H, O—CH₂—CH₂—CH₂—CH₂—NH—), 3.49-3.32 (m, 2H, O—CH₂—CH₂—CH₂—NH—), 1.83-1.77 (m, 2H, O—CH₂—CH₂—CH₂—NH—), 1.45 (s, 9H, C(CH₃)₃), 1.38 (s, 9H, C(CH₃)₃), 1.06-0.97 (m, 28H, tetraisopropyl-CH and —CH₃); HRMS (MALDI) was calculated to be 808.3955 for C₃₆H₆₃N₆O₁₁Si₂Na (M+Na⁺), and found to be 808.3991.

### 2′-O—(N,N′-Di-boc-guanidinopropyl)-uridine (3f)

To a solution of compound 3c (910 mg, 1.16 mmol) and triethylamine (240 μL) in 13 mL tetrahydrofurane NEt₃.3HF (700 μL, 4.3 mmol) was added. The reaction mixture was stirred for 1 h at room temperature. The solvents were evaporated and the residue was purified on a silica gel column using dichloromethane/methanol (93:7-92:8, v/v) to give 629 mg (97%) of a white foam. ¹H NMR (250 MHz, acetone-d₆) δ [ppm] 11.67 (bs, 1H, NH), 10.03 (bs, 1H, NH), 8.46-8.41 (m, 1H, NH—CH₂—), 8.10 (d, 1H, J=8.2 Hz, H6), 5.99-5.97 (m, 1H, H1′), 5.58 (d, 1H, J=8.2 Hz, H5), 4.39-3.46 (m, 11H), 1.95-1.85 (m, 2H, O—CH₂—CH₂—CH₂—NH—), 1.51 (s, 9H, C(CH₃)₃), 1.43 (s, 9H, C(CH₃)₃); ¹³C NMR (63 MHz, acetone-d₆) δ [ppm] 165.64, 164.59, 157.88, 154.86, 152.37, 142.33, 103.31, 89.82, 86.67, 84.77, 84.74, 84.39, 79.91, 70.73, 70.69, 62.45, 40.20, 30.96, 29.51, 29.19; MS (ESI) was calculated to be 566.2 for C₂₃H₃₇N₅O₁₀Na (M+Na⁺), and found to be 567.0.

### 2′-O—(N,N′-Di-boc-guanidinopropyl)-5′-O-(4,4′-dimethoxytrityl)-uridine (3g)

2′-O—(N,N′-Di-boc-guanidinopropyl)-uridine (3f) (588 mg, 1.08 mmol) was dissolved in 11.4 mL of dry pyridine and 4,4′-Dimethoxytrityl chloride (460 mg, 1.36 mmol) was added. The reaction solution was stirred at room temperature for 5 h. The reaction mixture was quenched with water and the solvents were evaporated. The residue was dissolved in dichloromethane, washed twice with saturated sodium bicarbonate solution (2×50 mL) and then twice with brine (2×50 mL). The organic layer was dried over Na₂SO₄ and the solvent was removed under reduced pressure. After purification using column chromatography with dichloromethane/methanol (97:3, v/v) containing 0.5% triethylamine, 785 mg (86%) of a yellow powder was obtained. The yellow impurity could not be separated on the column. ¹H NMR (250 MHz, DMSO-d₆) δ [ppm] 11.49 (s, 1H, NH), 11.37 (m, 1H, NH), 8.41-8.36 (m, 1H, NH—CH₂—), 7.75 (d, 1H, J=8.1 Hz, H6), 7.40-7.23 (m, 9H, DMTr), 6.92-6.88 (m, 4H, DMTr), 5.83-5.82 (m, 1H, H1′), 5.29-5.25 (m, 1H, H5), 5.09-5.06 (m, 1H, 3′-OH), 4.23-3.88 (m, 3H), 3.74 (s, 6H, 2×O—CH₃), 3.68-3.63 (m, 2H), 3.43-3.20 (m, 4H), 1.82-1.72 (m, 2H, O—CH₂—CH₂—CH₂—NH—), 1.44 (s, 9H, C(CH₃)₃), 1.37 (s, 9H, C(CH₃)₃); HRMS (MALDI) was calculated to be 846.3920 for C₄₄H₅₆N₅O₁₂ (M+H⁺), and found to be 846.3946.

### 2′-O—(N,N′-Di-boc-guanidinopropyl)-5′-O-(4,4′-dimethoxytrityl)-uridine 3′-(cyanoethyl)N,N-diisopropyl phosphoramidite (3d)

2′-O—(N,N′-Di-boc-guanidinopropyl)-5′-O-(4,4′-dimethoxytrityl)-uridine (3g) (770 mg, 0.9 mmol) was dissolved in dichloromethane (11 mL). To this solution, 2-cyanoethyl N,N,N′,N′-tetraisopropylamino phosphane (400 μL, 1.26 mmol) and 4,5-dicyanoimidazole (130 mg, 1.1 mmol) were added. The reaction progress was observed with TLC (dichloromethane/ethyl acetate 1:1 (v:v), containing 0.5% triethylamine). Because the reaction was not complete after two hours, an additional 0.3 equivalents of the reagents were added and the reaction was completed after additional 40 minutes. The resulting solution was washed twice with saturated sodium bicarbonate solution (2×100 mL) and once with brine (200 mL). After drying over Na₂SO₄ the solvent was evaporated and the residue was purified on a silica gel column with dichloromethane/ethyl acetate (6:4-1:1, v/v) containing 0.5% triethylamine. The mixture of the two diastereomers was obtained as a light yellow foam (762 mg, 83%). ¹H NMR (400 MHz, DMSO-d₆) δ [ppm] 11.50-11.48 (m, 1H, NH), 11.35 (bs, 1H, NH), 8.39-8.33 (m, 1H, NH—CH₂—), 7.87-7.80 (m, 1H, H6), 7.41-7.22 (m, 9H, DMTr), 6.91-6.86 (m, 4H, DMTr), 5.86-5.84 (m, 1H, H1′), 5.23-5.18 (m, 1H, H5), 4.46-4.32 (m, 1H), 4.21-4.16 (m, 1H), 4.09-4.03 (m, 1H), 3.83-3.26 (m, 16H), 2.80-2.59 (m, 2H, —O—CH₂—CH₂—CN), 1.81-1.74 (m, 2H, O—CH₂—CH₂—CH₂—NH—), 1.42-1.36 (m, 18H, C(CH₃)₃), 1.13-0.94 (m, 12H, iPr-CH₃); ³¹H NMR (121 MHz, DMSO-d₆) δ [ppm] 150.0, 148.6; HRMS (MALDI) was calculated to be 1046,4999 for C₆₃H₇₃N₇O₁₃P (M+H⁺), and found to be 1046,5021.

### Example 5

### Synthesis of the 2′-O-Guanidinopropyl Guanosine Phosphoramidite

O⁶-(2,4,6-Triisopropylbenzenesulfonyl)-3′,5′-O-di-tert-butylsilanediyl guanosine (4a) was synthesised according to a previously described procedure [21].

### 2′-O-(2-Cyanoethyl)-3′,5′-O-di-tert-butylsilanediyi guanosine (4b)

Compound 4a (2.28 g, 3.3 mmol) was dissolved in tert-butanol (17 mL). Freshly distilled acrylonitrile (4.25 mL, 66 mmol) and cesium carbonate (1.16 g, 3.3 mmol) were added to the solution. After vigorous stirring at room temperature for 2-3 h, the mixture was filtered through celite. The solvent and excess reagents were removed in vacuum. The crude material was used for the next reaction without further purification. The residue was dissolved in 4 mL of a mixture of formic acid/dioxane/water (70:24:6, v/v/v). After stirring at room temperature for 1 h, water (150 mL) was added to the mixture and the solution extracted with dichloromethane. The organic layer was dried over Na₂SO₄ and the solvent was evaporated. The residue was purified using column chromatography with dichloromethane/methanol (9:1, v/v) to give 1.1 g (70% over 2 steps) of 4b as a colourless foam. ¹H NMR (250 MHz, DMSO-d₆) δ [ppm] 10.71 (bs, 1H, NH), 7.89 (s, 1H, H8), 6.45 (bs, 2H, NH₂), 5.81 (s, 1H, H1′), 4.45-4.33 (m, 3H), 4.05-3.81 (m, 4H), 2.83-2.76 (m, 2H, O—CH₂—CH₂—CN), 1.06 (s, 9H, C(CH₃)₃), 1.01 (s, 9H, C(CH₃)₃); ¹³C NMR (63 MHz, DMSO-d₆) δ [ppm] 156.51, 153.69, 150.50, 135.36, 118.71, 116.53, 87.25, 80.31, 76.35, 73.80, 66.64, 65.14, 27.12, 26.80, 22.07, 19.82, 18.29; MS (ESI) was calculated to be 477.2 for C₂₁H₃₃N₆O₅Si (M+H⁺), and found to be 477.5.

### 2′-O-(2-Aminopropyl)-3′,5′-O-di-tert-butylsilanediyl guanosine (4e)

Compound 4b (500 mg, 1.06 mmol) was dissolved in dry methanol (5 mL). Raney nickel (ca. 0.5 mL of the methanol-washed sediment) and methanol (5 mL) saturated with ammonia were then added. The mixture was hydrogenated at 30 bar hydrogen-pressure for 1 h at room temperature. Thereafter the mixture was filtered through a glass filter and the catalyst was washed several times with methanol and a methanol/water mixture. The solvents were evaporated from the filtrate and the residue was used without further purification for the next reaction. MS (ESI) was calculated to be 481.3 for C₂₁H₃₇N₆O₅Si (M+H⁺), and found to be 481.8.

### 2′-O—(N,N′-Di-boc-guanidinopropyl)-3′,5′-O-di-tert-butylsilanediyl guanosine (4c)

N,N′-Di-boc-N″-triflyl guanidine (163 mg, 0.415 mmol) was dissolved in dichloromethane (2.1 mL) and triethylamine (54 μL) was then added. The solution was cooled in an ice bath and then 2′-O-(2-Aminopropyl)-3′,5′-O-di-tert-butylsilanediyl guanosine (4e) (200 mg, 0.42 mmol) was added. After 30 minutes the reaction mixture was removed from the ice bath then stirred for an additional 30 minutes at room temperature. The reaction solution was washed with saturated sodium bicarbonate solution and brine. After drying over Na₂SO₄ the solvent was evaporated. The residue was purified by column chromatography using dichloromethane/methanol (9:1, v/v) to give 270 mg (89%) of 4c. ¹H NMR (400 MHz, DMSO-d₆) δ [ppm] 11.49 (bs, 1H, NH), 10.66 (bs, 1H, NH), 8.56-8.53 (m, 1H, NH—CH₂—), 7.87 (s, 1H, H8), 6.39 (bs, 2H, NH₂), 5.86 (s, 1H, H1′), 4.42-4.38 (m, 1H, H3′), 4.30-4.27 (m, 2H, H2′, H5′), 4.06-3.93 (m, 3H, H4′, H5′, ½×O—CH₂—CH₂—CH₂—NH—), 3.72-3.67 (m, 1H, ½×O—CH₂—CH₂—CH₂—NH—), 3.51-3.30 (m, 2H, O—CH₂—CH₂—CH₂—NH—), 1.84-1.77 (m, 2H, O—CH₂—CH₂—CH₂—NH—), 1.46 (s, 9H, —CO—C(CH₃)₃), 1.39 (s, 9H, —CO—C(CH₃)₃), 1.06 (s, 9H, —Si—C(CH₃)₃), 0.97 (s, 9H, —Si—C(CH₃)₃); HRMS (MALDI) was calculated to be 723.3856 for C₃₂H₅₅N₅O₉Si (M+H⁺), and found to be 723.3880.

### N²-Isobutyryl-2′-O—(N,N′-di-boc-guanidinopropyl)-3′,5′-O-di-tert-butylsilanediyl guanosine and N²-Isobutyryl-2′-O—(N,N′-di-boc-N″-isobutyryl-guanidinopropyl)-3′,5′-O-di-tert-butylsilanediyl guanosine (4f)

A solution of compound 4c (400 mg, 0.55 mmol) in 3.6 mL of pyridine was cooled in an ice bath and isobutyryl chloride (145 μL, 1.37 mmol) was then added dropwise. The mixture was stirred at 0° C. for 1 h, then at room temperature for 1 h and evaporated to dryness. The residue was dissolved in 40 mL dichloromethane and washed twice with saturated sodium bicarbonate solution (2×60 mL) and once with brine (60 mL). The organic phase was dried over Na₂SO₄ and the solvent was evaporated. The residue was purified by column chromatography using dichloromethane/methanol (95:5-90:10, v/v) to give 318 mg (ca. 70%) of a mixture of mono- and di-isobutyryl derivative. ¹H NMR (250 MHz, DMSO-d₆) δ [ppm] 12.12 (s, 1H, NH), 11.57-11.51 (m, NH, NH-boc), 10.53 (s, NH-boc*), 8.54-8.49 (m, 2′-O—CH₂—CH₂—CH₂—NH—), 8.25-8.22 (m, 1H, H8), 5.90-5.88 (m, 1H, H1′), 4.42-3.42 (m, 9H), 2.85-2.72 (m, 1.5H, —CH(CH₃)₂), 1.99-1.73 (m, 2H, 2′-O—CH₂—CH₂—CH₂—NH—), 1.47-1.33 (m, 18H, 2×-CO—C(CH₃)₃), 1.15-0.99 (m, 27H, 2×—Si—C(CH₃)₃, —CH(CH₃)₂, —CH(CH₃)₂*). As a result of the mixture comprising mono- and diisobutyryl derivatives, some of the integrals could not be given as whole numbers. Thus, signals that depend only on the diisobutyryl compound are marked with an asterisk. MS (ESI) was calculated to be 793.4 for C₃₆HₑᵢN₈O₁₀Si (M+H⁺), and found to be 794.6.

### N²-Isobutyryl-2′-O—(N,N′-di-boc-guanidinopropyl)-guanosine and N²-Isobutyryl (N,N′-di-boc-N″-isobutyryl-guanidinopropyl)-guanosine (4g*)

A mixture of N²-Isobutyryl-2′-O—(N,N′-di-boc-guanidinopropyl)-3′,5′-O-di-tert-butylsilanediyl guanosine (4f) and N²-Isobutyryl-2′-O—(N,N′-di-boc-N″-isobutyryl-guanidinopropyl)-3′,5′-O-di-tert-butylsilanediyl guanosine (4f*) (490 mg, ca. 592 μmol) was dissolved in dry tetrahydrofurane (7 mL). Triethylamine (165 μL, 1.11 mmol) and Et₃N-3HF (352 μL, 2.16 mmol) were then added. After stirring at room temperature for 1 h the solvent was evaporated. The residue was purified using column chromatography with dichloromethane/methanol (9:1, v/v) to give 322 mg (ca. 79%) of a mixture of N²-Isobutyryl-2′-O(N,N′-di-boc-guanidinopropyl)-guanosine and N²-Isobutyryl-2′-O—(N,N′-di-boc-N″-isobutyryl-guanidinopropyl)-guanosine as white foam. A small sample of the mixture was separated for NMR spectroscopy. NMR data is given for the mono-isobutyryl compound. ¹H NMR (400 MHz, DMSO-d₆) δ [ppm] 12.08 (s, 1H, NH), 11.65 (s, 1H, NH), 11.46 (s, 1H, NH), 8.29 (s, 1H, H8), 8.28-8.25 (m, 1H, NH—CH₂—), 5.91 (d, 1H, J=6.0 Hz, H1′), 5.16 (d, 1H, J=4.8 Hz, 3′-OH), 5.06-5.03 (m, 1H, 5′-OH), 4.36-4.29 (m, 2H, H2′, H3′), 3.95-3.93 (m, 1H, H4′), 3.67-3.46 (m, 4H, 2×H5′, O—CH₂—CH₂—CH₂—NH—), 3.33-3.28 (m, 2H, O—CH₂—CH₂—CH₂—NH—), 2.77 (sep, 1H, J=6.8 Hz, —CH(CH₃)₂), 1.75-1.67 (m, 2H, O—CH₂—CH₂—CH₂—NH—), 1.45 (s, 9H, —CO—C(CH₃)₃), 1.37 (s, 9H, —CO—C(CH₃)₃), 1.12 (d, 6H, J=6.8 Hz, —CH(CH₃)₂); ¹³C NMR (63 MHz, CDCl₃) δ [ppm] 178.72, 163.52, 156.12, 155.16, 153.39, 147.73, 147.05, 138.81, 122.49, 88.47, 86.74, 83.65, 82.28, 79.58, 70.69, 69.87, 62.66, 38.87, 36.39, 29.32, 28.28, 28.11, 18.96, 18.89; HRMS (MALDI) was calculated to be 653.3253 for C₂₈H₄₅N₈O₁₀ (M+H⁺), and found to be 653.3274.

### N²-Isobutyryl-2′-O—(N,N′-di-boc-guanidinopropyl)-5′-O-(4,4′-dimethoxytrityl)-guanosine and N²-Isobutyryl-2′-O—(N,N′-di-boc-N″-isobutyryl-guanidinopropyl)-5′-O-(4,4′-dimethoxytrityl)-guanosine (4h*)

A mixture of N²-Isobutyryl-2′-O—(N,N′-di-boc-guanidinopropyl)-guanosine (4g) and N²-Isobutyryl-2′-O—(N,N′-di-boc-N″-isobutyryl-guanidinopropyl)-guanosine (4g*) (400 mg, ca. 583 μmol) was dissolved in dry pyridine (11 mL). 4,4′-Dimethoxytrityl chloride (280 mg, 0.82 mmol) was added and the solution was stirred for 3 h at room temperature. TLC revealed that some starting material remained at this time and an additional 0.3 equivalents of 4,4′-Dimethoxytrityl chloride were therefore added. When TLC demonstrated that the starting material had been consumed, the reaction was quenched with water and the solvents evaporated. The residue was purified by column chromatography using dichloromethane/methanol (98:2, v/v) containing 0.5% triethylamine to give 427 mg (ca. 74%) of the desired products. ¹H NMR (400 MHz, DMSO-d₆) δ [ppm] 12.09 (s, 1H, NH), 11.58 (s, 1H, NH), 11.47 (s, 0.5H, NH-boc), 10.50 (s, 0.5H, NH-boc*), 8.33-8.30 (m, 0.5H, 2′-O—CH₂—CH₂—CH₂—NH—), 8.15-8.12 (m, 1H, H8), 7.35-7.18 (m, 9H, DMTr), 6.84-6.80 (m, 4H, DMTr), 5.97-5.94 (m, 1H, H1′), 5.15-5.13 (m, 1H, 3′-OH), 4.42-4.37 (m, 1H, H2′), 4.35-4.30 (m, 1H, H3′), 4.09-4.03 (m, 1H, H4′), 3.72 (s, 6H, 2×O—CH₃), 3.69-3.47 (m, 2H, 2′-O—CH₂—CH₂—CH₂—NH—), 3.37-3.26 (m, 3H, 2′-O—CH₂—CH₂—CH₂—NH—, H5′), 3.17-3.13 (m, 1H, H5′), 2.79-2.73 (m, 1.5H, —CH(CH₃)₂), 1.77-1.67 (m, 2H, 2′-O—CH₂—CH₂—CH₂—NH—), 1.43-1.35 (m, 18H, 2×-CO—C(CH₃)₃), 1.13-1.10 (m, 6H, —CH(CH₃)₂), 1.00-0.98 (m, 3H, —CH(CH₃)₂*). As a result of the mixture comprising mono- and diisobutyryl derivatives, some of the integrals could not be given as whole numbers. Thus, signals that depend only on the diisobutyryl compound are marked with an asterisk. MS (ESI) was calculated to be 955.5 for C₄₉H₆₃N₈O₁₂ (M+H⁺), and found to be 956.5.

### N²-Isobutyryl-2′-O—(N,N′-di-boc-guanidinopropyl)-5′-O-(4,4′-dimethoxytrityl)-guanosine 3′-(cyanoethyl)-N,N-diisopropyl phosphoramidite (4d) and N²-Isobutyryl-2′-O—(N,N′-di-boc-N″-isobutyryl-guanidinopropyl)-5′-O-(4,4′-dimethoxytrityl)-guanosine 3′-(cyano ethyl)-N,N-diisopropyl phosphoramidite (4d*)

A mixture of N²-Isobutyryl-2′-O—(N,N′-di-boc-guanidinopropyl)-5′-O-(4,4′-dimethoxy trityl)-guanosine (4h) and N²-Isobutyryl-2′-O—(N,N′-di-boc-N″-isobutyryl-guanidinopropyl)-5′-O-(4,4′-dimethoxytrityl)-guanosine (4h*) (380 mg, ca. 384 μmol) was dissolved in dichloro methane (8 mL), then 2-cyanoethyl N,N,N′,N′-tetraisopropylamino phosphane (160 μL, 0.52 mmol) and 4,5-dicyanoimidazole (57 mg, 0.5 mmol) were added. After 2 h TLC showed complete consumption of the starting material. The reaction solution was then washed twice with saturated sodium bicarbonate solution (2×50 mL) and once with brine (100 mL). After drying over Na₂SO₄ the solvent was evaporated and the residue was purified using column chromatography with dichloromethane/ethyl acetate (8:2, v/v) containing 0.5% triethylamine to give 350 mg (ca. 76%) of the two diastereomers of 4d and 4d*. ¹H NMR (400 MHz, DMSO-d₆) δ [ppm] 12.11 (bs, 1H, NH), 11.61-11.57 (m, 1H, NH), 11.46-11.44 (m, 0.5H, NH-boc), 10.50-10.46 (m, 0.5H, NH-boc*), 8.31-8.27 (m, 0.5H, 2′-O—CH₂—CH₂—CH₂—NH—), 8.18-8.14 (m, 1H, H8), 7.36-7.19 (m, 9H, DMTr), 6.85-6.80 (m, 4H, DMTr), 5.97-5.88 (m, 1H, H1′), 4.64-4.61 (m, 1H, H2′), 4.44-4.37 (m, 1H, H3′), 4.27-4.12 (m, 1H, H4′), 3.79-3.18 (m, 10H), 3.72 (s, 6H, 2×OCH₃), 2.81-2.70 (m, 2.5H, —CH(CH₃)₂), 2.60-2.47 (m, 2H, —P—O—CH₂—CH₂—CN), 1.75-1.65 (m, 2H, 2′-O—CH₂—CH₂—CH₂—NH—), 1.41-1.34 (m, 18H, 2×-CO—C(CH₃)₃), 1.15-1.10 (m, 18H, —N((CH(CH₃)₂)₂, —CO—CH(CH₃)₂), 1.00-0.96 (m, 3H, —CH(CH₃)₂*); ³¹P NMR (162 MHz, DMSO-d₆) δ [ppm] 149.59, 149.44, 149.52, 149.19. As a result of the mixture comprising mono- and diisobutyryl derivatives, some of the integrals could not be given as whole numbers. Thus, signals that depend only on the diisobutyryl compound are marked with an asterisk. MS (ESI) was calculated to be 1155.6 for C₆₈H₆₀N₁₀O₁₃P (M+H⁺), and found to be 1157.3.

### Example 6

### Improved Synthesis of Guanosine phosphoramidite: 2′-O-guanidinopropyl-N²-dmf-guanosine phosphoramidite

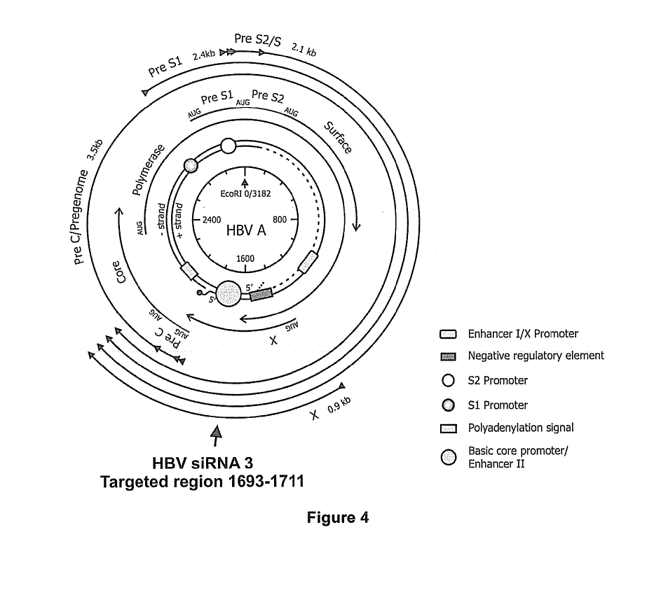

To circumvent the problem of a product mixture upon introduction of the isobutyryl group to the N²-position of guanosine, we established a different protection strategy, using the dimethylformamidine group (FIG. 3). The former synthesis yielded a mixture of mono- and di-isobutyryl compound but eventually after complete deprotection led to the desired RNA.

### N²-Dimethylformamidine-2′-O—(N,N′-di-boc-guanidinopropyl)-3′,5′-O-di-tert-butylsilanediyi guanosine

Compound 4c (1.12 g, 1.55 mmol) was dissolved in 25 mL dry methanol. N,N-Dimethylformamide dimethyl acetal (1.0 mL, 7.76 mmol) was added and the solution was stirred at room temperature overnight. After a reaction time of 12 h the solvents were removed in vacuum and the residue was purified by column chromatography using dichloromethane/methanol (19:1, v/v) to give 1.14 g (94%) of the dmf-protected derivative. ¹H NMR (400 MHz, DMSO-d₆) δ [ppm] 11.51 (s, 1H, N¹H), 11.40 (s, 1H, NH-boc), 8.54 (s, 1H, —N═CH—N(CH₃)₂), 8.47 (m, 1H, 2′-O—CH₂—CH₂—CH₂—NH—), 7.99 (s, 1H, H-8), 5.98 (s, 1H, H1′), 4.48-4.45 (m, 1H, H5′), 4.41-4.39 (m, 1H, H2′), 4.33-4.30 (m, 1H, H5′), 4.07-3.99 (m, 2H, H3′ und H4′), 3.98-3.77 (m, 2H, 2′-O—CH₂—CH₂—CH₂—NH—), 3.48-3.37 (m, 2H, 2′-O—CH₂—CH₂—CH₂—NH—), 3.14 (s, 3H, N—CH₃), 3.04 (s, 3H, N—CH₃), 1.87-1.78 (m, 2H, 2′-O—CH₂—CH₂—CH₂—NH—), 1.47 (s, 9H, —CO—C(CH₃)₃), 1.37 (s, 9H, —CO—C(CH₃)₃), 1.06 (s, 9H, —Si—C(CH₃)₃), 1.00 ppm (s, 9H, —Si—C(CH₃)₃); ¹³C NMR (75 MHz, DMSO-d₆) δ [ppm] 163.00, 157.60, 157.39, 157.35, 154.98, 151.95, 149.21, 136.96, 119.86, 88.07, 82.78, 80.59, 77.90, 76.48, 73.83, 69.64, 66.77, 44.41, 40.58, 34.54, 28.61, 27.86, 27.44, 27.08, 26.70, 22.06, 19.76; HRMS (MALDI) was calculated to be 800.4097 for C₃₅H₅₉N₉O₉SiNa (M+Na⁺), and found to be 800.4124.

### N²-Dimethylformamidine-2′-O—(N,N′-di-boc-guanidinopropyl)-guanosine

N²-Dimethylformamidine-2′-O—(N,N′-di-boc-guanidinopropyl)-3′,5′-O-di-tert-butylsilanediyl guanosine (1.24 g, 1.59 mmol) was dissolved in dry tetrahydrofurane (17 mL). Triethylamine (470 μL, 3.18 mmol) and Et₃N.3HF (943 μL, 5.79 mmol) were then added. After stirring at room temperature for 1 h the solvent was evaporated. The residue was purified using column chromatography with dichloromethane/methanol (9:1, v/v) to give 840 mg (83%) of N²-Dimethylformamidine-2′-O—(N,N′-di-boc-guanidinopropyl)-guanosine as white foam. ¹H NMR (400 MHz, DMSO-d₆) δ [ppm] 11.50 (s, 1H, N¹H), 11.34 (s, 1H, NH-boc), 8.54 (s, 1H, —N═CH—N(CH₃)₂), 8.35 (m, 1H, 2′-O—CH₂—CH₂—CH₂—NH—), 8.10 (s, 1H, H8), 5.95-5.94 (m, 1H, H1′), 5.14-5.12 (m, 1H, 3′-OH), 5.08-5.05 (m, 1H, 5′-OH), 4.31-4.30 (m, 2H, H2′, H3′), 3.95-3.93 (m, 1H, H4′), 3.67-3.56 (m, 4H, 2×H5′, O—CH₂—CH₂—CH₂—NH—), 3.36-3.33 (m, 2H, O—CH₂—CH₂—CH₂—NH—), 3.16 (s, 3H, N—CH₃), 3.04 (s, 3H, N—CH₃), 1.77-1.74 (m, 2H, O—CH₂—CH₂—CH₂—NH—), 1.47 (s, 9H, —CO—C(CH₃)₃), 1.37 (s, 9H, —CO—C(CH₃)₃); ¹³C NMR (75 MHz, DMSO-d₆) δ [ppm] 162.98, 157.84, 157.44, 157.24, 155.10, 151.89, 149.61, 136.41, 119.77, 85.26, 85.23, 82.75, 81.36, 78.00, 68.51, 67.87, 60.81, 40.54, 37.86, 34.53, 28.65, 27.85, 27.50; HRMS (MALDI) was calculated to be 660.3076 for O₂₇H₄₃N₉O₉Na (M+Na⁺), and found to be 660.3087.

### N²-Dimethylformamidine-2′-O—(N,N′-di-boc-guanidinopropyl)-5′-O-(4,4′-dimethoxytrityl)-guanosine

N²-Dimethylformamidine-2′-O—(N,N′-di-boc-guanidinopropyl)-guanosine (840 mg, 1.32 mmol) was dissolved in dry pyridine (30 mL). 4,4′-Dimethoxytrityl chloride (670 mg, 1.98 mmol) was added and the solution was stirred for 3 h at room temperature. After the reaction was complete according to TLC, the reaction was quenched with methanol and the solvents were evaporated. The residue was purified by column chromatography using dichloromethane/methanol (100:0->95:5, v/v; the column was packed with dichloromethane containing 0.5% triethylamine) to give 1.08 g (87%) of the desired product. ¹H NMR (400 MHz, DMSO-d₆) δ [ppm] 11.51 (s, 1H, N¹H), 11.38 (s, 1H, NH-boc), 8.50 (s, 1H, —N═CH—N(CH₃)₂), 8.40 (m, 1H, 2′-O—CH₂—CH₂—CH₂—NH—), 7.94 (s, 1H, H8), 7.38-7.20 (m, 9H, DMTr), 6.86-6.82 (m, 4H, DMTr), 6.01-6.00 (m, 1H, H1′), 5.16-5.13 (m, 1H, 3′-OH), 4.35-4.30 (m, 2H, H2′, H3′), 4.08-4.05 (m, 1H, H4′), 3.73 (s, 6H, 2×O—CH₃), 3.71-3.61 (m, 2H, 2′-O—CH₂—CH₂—CH₂—NH—), 3.40-3.35 (m, 2H, 2′-O—CH₂—CH₂—CH₂—NH—), 3.28-3.16 (m, 2H, 2×H5′), 3.09 (s, 3H, N—CH₃), 3.02 (s, 3H, N—CH₃), 1.80-1.74 (m, 2H, 2′-O—CH₂—CH₂—CH₂—NH—), 1.44 (s, 9H, —CO—C(CH₃)₃), 1.34 (s, 9H, —CO—C(CH₃)₃); ¹³C NMR (100 MHz, DMSO-d₆) δ [ppm] 162.96, 157.97, 157.95, 157.72, 157.48, 157.20, 155.10, 151.89, 149.59, 144.71, 136.15, 135.43, 135.30, 129.59, 129.57, 127.67, 127.57, 126.55, 119.83, 113.02, 85.53, 85.37, 82.72, 82.69, 81.04, 77.96, 69.02, 68.22, 63.55, 54.90, 40.54, 37.99, 34.54, 28.66, 27.82, 27.49; HRMS (MALDI) was calculated to be 962.4383 for C₄₈H₆₁N₉O₁₁Na (M+Na⁺), and found to be 962.4408.

### N²-Dimethylformamidine-2′-O—(N,N′-di-boc-guanidinopropyl)-5′-O-(4,4′-dimethoxytrityl)guanosine 3′-(cyanoethyl)-N,N-diisopropyl phosphoramidite

N²-Dimethylformamidine-2′-O—(N,N′-di-boc-guanidinopropyl)-5′-O-(4,4′-dimethoxytrityl)-guanosine (1.22 g, 1.3 mmol) was dissolved in dichloromethane (25 mL), then 2-cyanoethyl N,N,N′,N′-tetraisopropylamino phosphane (590 μL, 1.76 mmol) and 4,5-dicyanoimidazole (199 mg, 1.69 mmol) were added. After 4 h TLC showed complete consumption of the starting material. The reaction solution was then washed twice with saturated sodium bicarbonate solution and once with brine. After drying over Na₂SO₄ solvent was evaporated and the residue was purified using column chromatography with dichloromethane/acetone/methanol (4:0:1->3:0:2->2:1:2->2:2:1, v/v, the column was packed with eluent containing 0.5% triethylamine). The residue was dissolved in a small amount (5 mL) of dichloromethane. This solution was dripped into a flask with hexane (500 mL) to form a white precipitate. Two thirds of the solvent was evaporated and the residual solvent was decanted carefully. The precipitate was redissolved in benzene and lyophilised to give 1.01 mg (68%) of the phosphoramidite. According to ³¹P NMR spectrum the product was still containing a small amount of the hydrolysed phosphitylation reagent but this did not interfere with the oligonucleotide synthesis. ¹H NMR (400 MHz, DMSO-d₆) δ [ppm] 11.50-11.48 (m, 1H, NH), 11.38 (G, 1H, NH), 8.44-8.42 (m, 1H, —N═CH—N(CH₃)₂), 8.39-8.34 (m, 1H, 2′-O—CH₂—CH₂—CH₂—NH—), 7.96 (s, 1H, H8), 7.37-7.19 (m, 9H, DMTr), 6.85-6.78 (m, 4H, DMTr), 6.07-6.05 (m, 1H, H1′), 4.64-4.58 (m, 1H, H3′), 4.48-4.44 (m, 1H, H2′), 4.26-4.19 (m, 1H, H4′), 3.80-3.23 (m, 10H), 3.73-3.70 (m, 6H, 2×OCH₃), 3.07 (s, 3H, N—CH₃), 3.02 (s, 3H, N—CH₃), 2.77-2.74 (m, 1H, —P—O—CH₂—CH₂—CN), 2.55-2.52 (m, 1H, —P—O—CH₂—CH₂—CN), 1.80-1.72 (m, 2H, 2′-O—CH₂—CH₂—CH₂—NH—), 1.44-1.34 (m, 18H, 2×-CO—C(CH₃)₃), 1.20-0.93 (m, 12H, —N((CH(CH₃)₂)₂); ³¹P NMR (121 MHz, DMSO-d₆) δ [ppm] 149.21, 148.93.

We also established an alternative reduction procedure according to a literature procedure [30].

This procedure was tested with 2′-O-(Cyanomethyl)-3′,5′-O-(tetraisopropyldisiloxane-1,3-diyl)uridine. 2′-O-(Cyanomethyl)-3′,5′-O-(tetraisopropyldisiloxane-1,3-diyl)uridine and 1 equivalent of dried (waterfree) Ni(II)Cl₂ was dissolved in absolute ethanol. 6 equivalents of sodium borohydride were added in small portions. After 4 h the reaction was quenched with 10 equivalents of diethylene triamine. The solvents were evaporated and the residue was dissolved in ethyl acetate. The solution was washed with saturated sodium bicarbonate solution and dried over MgSO₄. After evaporation of the solvent the residue was purified by column chromatography using dichloromethane/methanol (5:1, v/v) to give 2′-O-Aminoethyl-3′,5′-O-(tetraisopropyldisiloxane-1,3-diyl) uridine (ca. 30%).

### Example 7

### Oligonucleotide Synthesis

The obtained phosphoramidites where used for synthesis of the GP-modified siRNA antisense strands depicted in Table 1 and 2, and for the synthesis of GP-modified siRNA sense strands depicted in Table 3.

Modified oligonucleotides were synthesised on an Expedite 8909 synthesiser using phosphoramidite chemistry. The 2′-O-guanidinopropyl-modified nucleosides were inserted into the HBV antisense strand (intended guide, 5′-UUG AAG UAU GCC UCA AGG UCG-3′) (SEQ ID NO: 1) at each of positions 2, 3, 4, 5, 6, 7, 8, 9, 10, 11, 12, 13, 14, 15, 16, 17, 18, 19, 20 and 21 from the 5′ end (Table 1). In some antisense oligonucleotides, a combination of two (positions 2 & 5, 2 & 3, and 19 & 20), three (positions 2, 5 & 17 and 2, 3 & 17) or four (positions 2, 5, 17 & 20) 2′-O-guanidinopropyl-modifications was included (Table 2). The sense strand oligonucleotide 5′-ACC UUG AGG CAU ACU UCA AdTdT-3′ (SEQ ID NO: 2) included a single 2′-O-guanidinopropyl-modification at positions 17 or a combination of three 2′-O-guanidinopropyl-modification at positions 5, 13 and 17 (Table 3). The duplex HBV siRNA3 targeted HBV genotype A coordinates 1693 to 1711 (FIG. 4). Control siRNA with scrambled unmodified sequences comprised 5′-UAU UGG GUG UGC GGU CAC GGdT-3′ (antisense) (SEQ ID NO: 3) and 5′-CGU GAC CGC ACA CCC MU AdTdT-3′ (sense) (SEQ ID NO: 4). 5-Ethylthio-1H-tetrazole (0.25 M in can) was used as activator. Unmodified 2′-TBDMS-phorphoramidites were benzoyl- (A), isobutyryl- (G) or acetyl- (C) protected. Coupling time for the modified phosphoramidites was 25 minutes. After completion of synthesis, 30 minutes of deprotection in 3% trichloroacetic acid in dichloromethane was carried out to ensure complete cleavage of the boc groups. The RNA oligomers were cleaved from the controlled-pore-glass (CPG) support by incubation at 40° C. for 24 h using an ethanol:ammonia solution (1:3). The 2′-TBDMS groups were deprotected by incubation for 90 min at 65° C. with a triethylamine, N-methylpyrrolidinone and Et₃N.3HF mixture. RNA oligomers were precipitated with BuOH at 80° C. for 30 min and purified by anion exchange HPLC using a Dionex DNA-Pac 100 column. The oligonucleotides were desalted in a subsequent reverse phase HPLC step. Identity was confirmed by mass spectroscopy on a Bruker micrOTOF-Q.

TABLE 1
Single 2′-O-guanidinopropyl (GP) modified antisense synthesized
oligonucleotides, indicating the GP modified bases by (_{GP}) in
subscript.
Single antisense GP-modified siRNAs

| Name | Sequence |
|---|---|
| GP 2 siRNA3 | 5′-UU_{GP}G AAG UAU GCC UCA AGG UCG-3′ (SEQ ID NO: 5) |
| GP 3 siRNA3 | 5′-UUG_{GP} AGG UAU GCC UCA AGG UCG-3′ (SEQ ID NO: 6) |
| GP 4 siRNA3 | 5′-UUG A_{GP}AG UAU GCC UCA AGG UCG-3′ (SEQ ID NO: 7) |
| GP 5 siRNA3 | 5′-UUG AA_{GP}G UAU GCC UCA AGG UCG-3′ (SEQ ID NO: 8) |
| GP 6 siRNA3 | 5′-UUG AGG_{GP} UAU GCC UCA AGG UCG-3′ (SEQ ID NO: 9) |
| GP 7 siRNA3 | 5′-UUG AGG U_{GP}AU GCC UCA AGG UCG-3′ (SEQ ID NO: 10) |
| GP 8 siRNA3 | 5′-UUG AAG UA_{GP}U GCC UCA AGG UCG-3′ (SEQ ID NO: 11) |
| GP 9 siRNA3 | 5′-UUG AAG UAU_{GP} GCC UCA AGG UCG-3′ (SEQ ID NO: 12) |
| GP 10 siRNA3 | 5′-UUG AAG UAU G_{GP}CC UCA AGG UCG-3 (SEQ ID NO: 13) |
| GP 11 siRNA3 | 5′-UUG AAG UAU GC_{GP}C UCA AGG UCG-3′ (SEQ ID NO: 14) |
| GP 12 siRNA3 | 5′-UUG AAG UAU GCC_{GP} UCA AGG UCG-3′ (SEQ ID NO: 15) |
| GP 13 siRNA3 | 5′-UUG AAG UAU GCC U_{GP}CA AGG UCG-3′ (SEQ ID NO: 16) |
| GP 14 siRNA3 | 5′-UUG AAG UAU GCC UC_{GP}A AGG UCG-3′ (SEQ ID NO: 17) |
| GP 15 siRNA3 | 5′-UUG AAG UAU GCC UCA_{GP} AGG UCG-3′ (SEQ ID NO: 18) |
| GP 16 siRNA3 | 5′-UUG AAG UAU GCC UCA A_{GP}GG UCG-3′ (SEQ ID NO: 19) |
| GP 17 siRNA3 | 5′-UUG AAG UAU GCC UCA AG_{GP}G UCG-3′ (SEQ ID NO: 20) |
| GP 18 siRNA3 | 5′-UUG AAG UAU GCC UCA AGG_{GP} UCG-3′ (SEQ ID NO: 21) |
| GP 19 siRNA3 | 5′-UUG AAG UAU GCC UCA AGG U_{GP}CG-3′ (SEQ ID NO: 22) |
| GP 20 siRNA3 | 5′-UUG AAG UAU GCC UCA AGG UC_{GP}G-3′ (SEQ ID NO: 23) |
| GP 21 siRNA3 | 5′-UUG AAG UAU GCC UCA AGG UCG_{GP}-3′ (SEQ ID NO: 24) |

TABLE 2
Multiple 2′-O-guanidinopropyl (GP) modified antisense synthesised oligonucleo-
tides, indicating the GP modified bases by (_{GP}) in subscript.
Multiple GP-modified antisense siRNAs

| Name | Sequence |
|---|---|
| GP 2, 5 siRNA3 | 5′-UU_{GP}G AA_{GP}G UAU GCC UCA AGG UCG-3′ (SEQ ID NO: 25) |
| GP 2, 5, 17 siRNA3 | 5′-UU_{GP}G AA_{GP}G UAU GCC UCA AG_{GP}G UCG-3′ (SEQ ID NO: 26) |
| GP 2, 3 siRNA3 | 5′-UU_{GP}G_{GP} AAG UAU GCC UCA AGG UCG-3′ (SEQ ID NO: 27) |
| GP 2, 3, 17 siRNA3 | 5′-UU_{GP}G_{GP} AAG UAU GCC UCA AG_{GP}G UCG-3′ (SEQ ID NO: 28) |
| GP 19, 20 siRNA3 | 5′-UUG AAG UAU GCC UCA AGG U_{GP}C_{GP}G-3′ (SEQ ID NO: 29) |
| GP 2, 5, 17, 20 siRNA3 | 5′-UU_{GP}G AA_{GP}G UAU GCC UCA AG_{GP}G UC_{GP}G-3′ (SEQ ID NO: 30) |

TABLE 3
Single and multiple 2′-O-guanidinopropyl (GP) modified antisense synthesised
oligonucleotides, indicating the GP modified bases by GP in subscript.
GP-modified sense siRNA

| Name | Sequence |
|---|---|
| S GP 17 siRNA3 | 5′-ACC UUG AGG CAU ACU UC_{GP}A AdTdT-3′ (SEQ ID NO: 31) |
| S GP 5, 13, 17 siRNA3 | 5′-ACC UU_{GP}G AGG CAU A_{GP}CU UC_{GP}A AdTdT-3′ (SEQ ID NO: 32) |

### Example 8

### Inhibition of Firefly Luciferase Activity in Transfected Cells

Initially, to measure knockdown efficiency of 2′-O-guanidinopropyl-modified siRNAs in situ, HEK293 cells were co-transfected with RNAi activators together with a reporter gene plasmid (psiCHECK-HBx) [20] (FIG. 5). The siRNAs targeted a single sequence of the X open reading frame (ORF) of HBV (HBx) that has previously been shown to be an effective cognate for RNAi-based silencing [27]. Each of the siRNAs differed with respect to location of the 2′-O-guanidinopropyl modification, and these were within the seed region or at nucleotide 13 of the antisense strand of the siRNA duplex. siRNAs have been named according to the positioning of the 2′-O-guanidinopropyl (GP) modifications from the 5′ end of the intended guide strand. In psiCHECK-HBx, the viral target sequence was located in the Renilla transcript but downstream of the reporter ORF (FIG. 5A). Expression of Firefly luciferase is constitutively active to enable correction for variations in transfection efficiency. The ratio of Renilla to Firefly luciferase activity was used to assess knockdown efficacy. Compared to a scrambled siRNA control, analysis showed that the Firefly luciferase activity was diminished by approximately 70% when co-transfected with the unmodified siRNA (FIG. 5B). There was some variation in the efficacy of the inhibition of reporter gene activity that was dependent on the position of the chemically modified siRNAs. Knockdown efficacy was weakest with GP2 siRNA3, when the GP modification was placed at nucleotide 2 of the siRNA antisense sequence. siRNAs with the modification at positions 5 and 6 (GP5 siRNA3 and GP6 siRNA3) achieved most effective knockdown of reporter gene expression that was similar to that of the unmodified siRNA. A siRNA with the 2′-O-guanidinopropyl modification placed outside of the seed region at nucleotide 13 also achieved knockdown of 75%. 2′-O-guanidinopropyl modifications in anti HBV siRNA sequences are therefore compatible with effective target silencing, but position within the seed of the antisense guide influences efficacy.

### Cell Culture, Transfection, Dual Luciferase Assay and Measurement of HBV Surface Antigen (HBsAg) Concentrations.

Huh7 and HEK293 cells were cultured in DMEM (Lonza, Basel, Switzerland) supplemented with 5% foetal calf serum (Gibco BRL, UK). Cells were seeded in 24-well plates at a confluency of 40% on the day before transfection, and were then maintained in antibiotic-free medium for at least an hour prior to transfection. To assess target knockdown when using the luciferase reporter assay, Lipofectamine 2000 (Invitrogen, Carlsbad, Calif.) was employed to transfect HEK293 cells with 100 ng psiCHECK-HBx [20] and 32.5 ng siRNA (5 nM final concentration) at ratios of 1:1 and 1:3 (ml:mg) respectively. The psiCHECK-HBx reporter vector contains the HBx target sequence downstream of the Renilla ORF within the psiCHECK 2.2 (Promega, Wis., USA) and has been described previously [20]. Forty-eight hours after transfection, cells were assayed for luciferase activity using the Dual-Luciferase® Reporter Assay System (Promega, Wis., USA) and the ratio of Renilla luciferase to Firefly luciferase activity was calculated. Similarly, to assess knockdown of HBV replication in a liver-derived line, Huh7 cells were transfected with 100 ng pCH-9/3091 [28] and 32.5 ng siRNA. Forty eight hours after transfection, growth medium was harvested and HBsAg concentration was measured by ELISA using the MONOLISA® HBs Ag ULTRA kit (Bio-Rad, CA, USA). Each experiment was repeated at least in triplicate.

### Statistical Analysis

Data have been expressed as the mean±standard error of the mean. Statistical difference was considered significant when P<0.05 and was determined according to the student's t-test and calculated with the GraphPad Prism software package (GraphPad Software Inc., CA, USA).

### Example 9

### Inhibition of HBV Surface Antigen (HBsAg) Secretion from Transfected Cells by 2′-O-Guanidinopropyl-Modified siRNAs

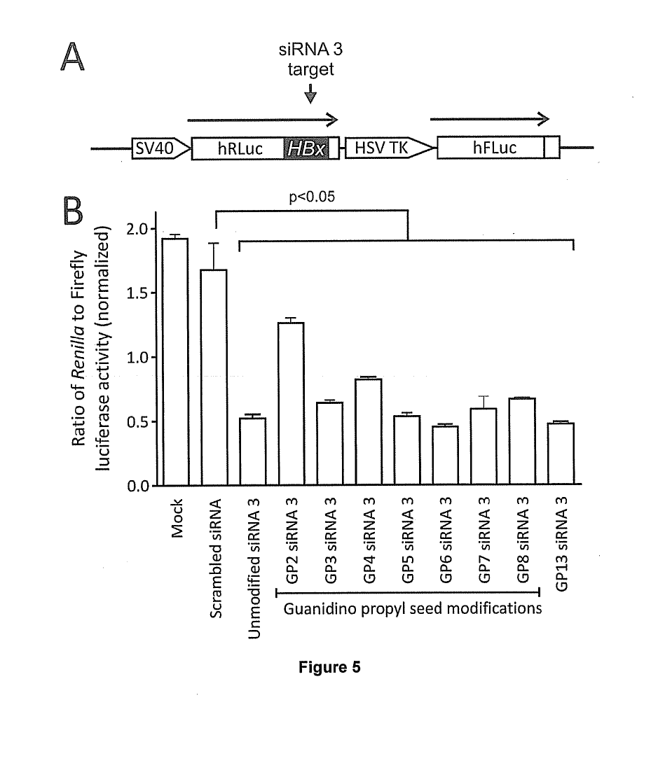

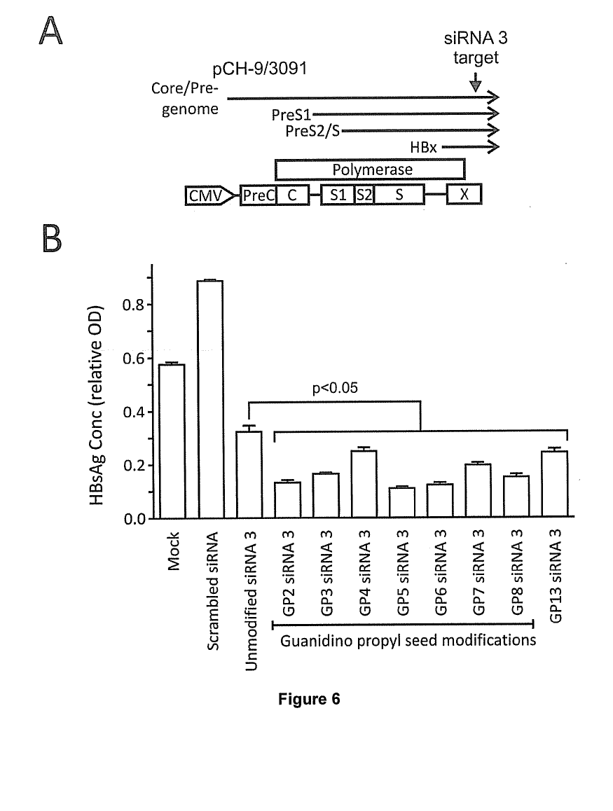

To assess efficacy against HBV replication in vitro, Huh7 liver-derived cells were co-transfected with siRNAs together with the pCH-9/3091 HBV replication competent target plasmid (FIG. 6A) [28]. Compared to HBsAg concentration in the culture supernatant of cells treated with scrambled siRNA, knockdown of up to 85% of viral antigen secretion was achieved by 2′-O-guanidinopropyl-modified siRNAs (FIG. 6B). The unmodified siRNA was slightly less effective than the siRNAs containing 2′-O-guanidinopropyl moieties. Of the modified siRNAs, positioning of the 2′-O-guanidinopropyl residue at nucleotides 5 or 6 (GP5 siRNA3 and GP6 siRNA3) resulted in the most effective suppression of HBsAg secretion (approximately 90%). These data correlate with observations using the reporter gene knockdown assay. Interestingly, GP2 siRNA3 inhibited HBsAg secretion from transfected cells more effectively than it did Renilla luciferase activity. The reason for this difference is unclear but may result from better GP2 siRNA3 target accessibility in the context of the natural HBV transcripts. Overall, these data support the notion that seed region GP modifications are compatible with effective target silencing that is similar or more effective than unmodified siRNAs.

### Example 10

### Stability of 2′-O-Guanidinopropyl-Modified siRNAs in 80% FCS

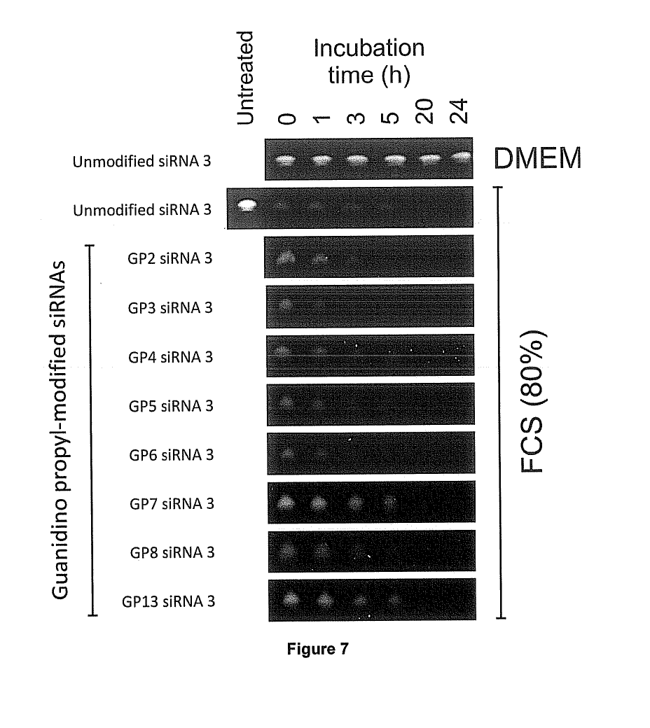

siRNAs containing 2′-O-guanidinopropyl (GP) modifications were incubated in the presence or absence of 80% fetal calf serum (FCS) for time intervals of 0 to 24 hours to assess their stability (FIG. 7). During the time course aliquots were removed and snap frozen using liquid nitrogen. Twenty picomoles of the samples were subjected to electrophoresis through a 10% denaturing polyacrylamide gel then stained with ethidium bromide. Bands corresponding to siRNAs were quantified to determine stability and FCS resistance. Analysis revealed that unmodified siRNA3 was stable for 24 hours when maintained in DMEM tissue culture medium that did not include FCS. However, rapid degradation of siRNA occurred in the presence of FCS, and approximately 18% of the input siRNA remained after 1 hour of incubation with FCS. Analysis of stability of GP2 siRNA3, GP3 siRNA3, GP4 siRNA3, GP5 siRNA3 and GP6 siRNA3 showed a slower degradation rate. For these modified siRNAs, 50-84% of the starting material was present after 1 hour's incubation with FCS. When the GP modifications were placed further from the 5′ end of the sense strand of the siRNA (GP7 siRNA3, GP8 siRNA3 and GP13 siRNA3) further stability of the siRNAs was conferred. With these siRNAs, 84-97% of starting material was present after 1 hour of incubation then 47-57% was intact after 5 hours' incubation. Stability is therefore improved by including GP modifications, but location of these moieties to central regions of the siRNAs is important to confer this property.

### Example 11

### Testing for Induction of the Non-Specific Interferon Response by Anti-HBV siRNA Sequences

Cell culture, transfection and RNA extraction. HEK293 cells were cultured and transfected as described previously. Briefly, cells were maintained in DMEM supplemented with 10% FCS, penicillin (50 IU/ml) and streptomycin (50 μg/ml) (Gibco BRL, UK). On the day prior to transfection, 250 000 HEK293 cells were seeded in dishes of 2 cm diameter. Transfection was carried out with 800 ng of unmodified or GP-containing siRNA using Lipofectamine (Invitrogen, CA, USA) according to the manufacturer's instructions. As a positive control for the induction of the interferon (IFN) response, cells were also transfected with 800 ng poly (I:C) (Sigma, Mich., USA). Two days after transfection, RNA was extracted with Tri Reagent (Sigma, Mich., USA) according to the manufacturer's instructions.

Real Time Quantitative PCR of Interferon Response Genes.

To assess induction of the interferon (IFN) response genes, IFN-β and GAPDH cDNA preparation and amplification where performed according to the procedures described by Song et al [31]. All qPCRs were carried out using the Roche Lightcycler V.2. Controls included water blanks and RNA extracts that were not subjected to reverse transcription. Tag readymix with SYBR green (Sigma, Mo., USA) was used to amplify and detect DNA during the reaction. Thermal cycling parameters consisted of a hotstart for 30 sec at 95° C. followed by 50 cycles of 58° C. for 10 sec, 72° C. for 7 sec and then 95° C. for 5 sec. Specificity of the PCR products was verified by melting curve analysis and agarose gel electrophoresis. The primer combinations used to amplify IFN-β mRNA and GAPDH mRNA of human HEK293 cells are set out in Table 4.

TABLE 4
Primers used to test for induction of the non-specific interferon
response by anti-HBV siRNA sequences.

| Primer Name | Sequence |
|---|---|
| IFN-β Forward | 5′-TCC AAA TTG CTC TCC TGT TGT GCT-3′ (SEQ ID NO: 33) |
| IFN-β Reverse | 5′-CCA CAG GAG CTT CTG ACA CTG AAA A-3′ (SEQ ID NO: 34) |
| GAPDH Forward | 5′-AGG GGT CAT TGA TGG CAA CAA TAT CCA-3′ (SEQ ID NO: 35) |
| GAPDH Reverse | 5′-TTT ACC AGA GTT AAA AGC AGC CCT GGT G-3′. (SEQ ID NO: 36) |

Interferon Response Gene Induction in Transfected Cells.

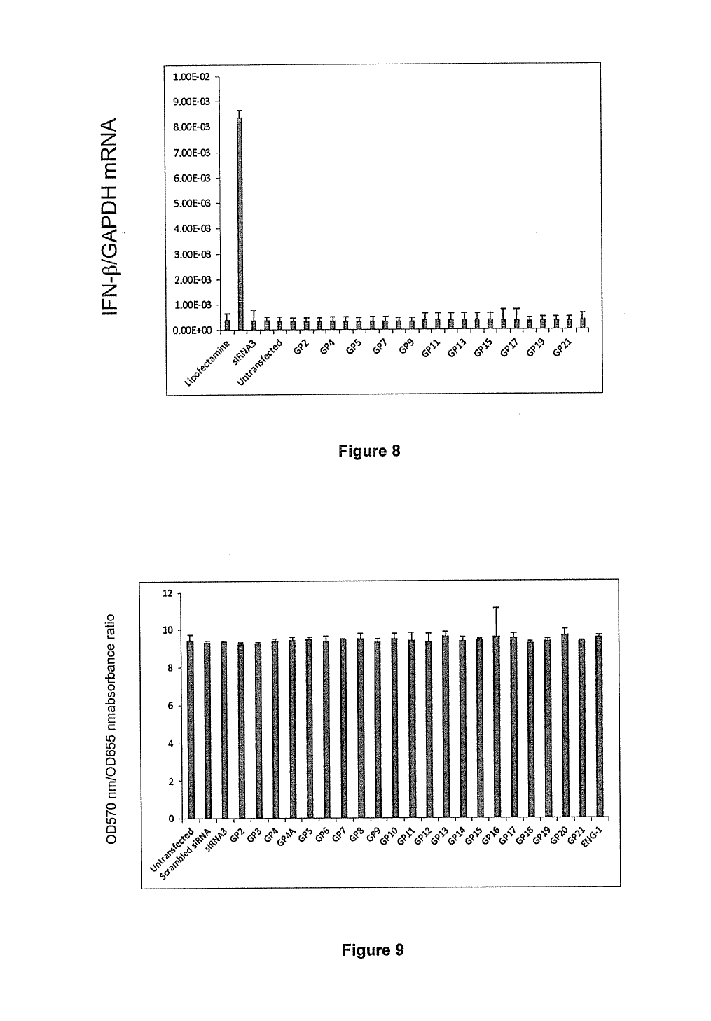

FIG. 8 shows a comparison of the concentration ratio of IFN-β mRNA to GAPDH mRNA, which is a housekeeping gene. Expression of IFN-β was increased at 24 hours after treatment of cells with poly (I:C), which confirms activation of the IFN response under the experimental conditions used here. Induction of IFN-β mRNA was not observed with RNA extracted from cells that had been transfected any of the unmodified or GP-containing siRNAs. These data indicate that the silencing effect of siRNAs on HBV markers of replication is unlikely to result from a non-specific induction of the interferon response.

### Example 12

### Influence of GP Modifications of siRNAs on Cell Viability Using the MTT Assay

The principle of the sensitive cell viability assay is based on conversion of the yellow 3-(4,5-dimethylhiazol-2-yl)-2,5-diphenyl tetrazolium bromide (MTT) to a dark blue/purple product by mitochondrial succinyl dehydrogenase. The insoluble product is solubilised in a solvent (dimethylsyulphoxide, DMSO) and the concentration measured spectrophotometrically by determining the optical density ratio at 570 nm, which shows the concentration of MTT product, to that at 655 nm, which indicates the number of cells that was analysed in each assay. Since conversion of the substrate to product can only occur in metabolically active cells, the activity of the mitochondrial dehydrogenase can be used conveniently as a measure of cell viability. Procedures were followed according to the recommendations of the supplier of the MTT (Sigma, Mich., USA). MTT cells were plated in 125 μl media per well in a 96-well plate, then incubated overnight (at 37° C., 5% CO₂) to allow cells to attach. Cells were then transfected with unmodified or GP-modified siRNAs (2.5 nM or 8.125 ng per well) and gently mixed by shaking. Cells were then cultured for a further 1-5 days. MTT substrate (Sigma, Mich., USA) was freshly prepared by dissolving in 1×Dulbecco's Phosphate Buffered Saline (DPBS) or Phosphate Buffered Saline (PBS) at a concentration of 5 mg/ml. Twenty μl of the MTT solution was added to each well and gently mixed for 5 minutes. Thereafter the plates were incubated for a further 1-5 hours to allow metabolism of MTT. The medium was then gently removed from each well. The blue MTT metabolic product, formazan, was resuspended in 200 μl DMSO and gently mixed by shaking for 5 minutes. The spectrophotometric optical density was measured at 570 nm and divided by the background reading at 655 nm.

FIG. 9 shows a comparison of spectrophotometrically detected OD 570 nm/OD 655 nm ratios indicating the amounts of product generated after solubilisation. The results indicate that there is no significant difference between the cells that had been treated with GP-modified siRNAs and the control untransfected cells. This indicates that the modified siRNAs do not have any detectable toxic effect on cells.

### Example 13

### Influence of the Position of the GP Modification on Silencing of Complete and Partial HBV Targets Using the Dual Luciferase Reporter Assay

A panel of dual luciferase reporter plasmids was generated in which the HBV target sequences included variable numbers of nucleotides that were complementary to the intended siRNA3 guide strand. The targets in the reporter plasmids are listed below:
1. Complete target (CT), complete base complementarity between target HBV and siRNA3 guide.
2. Incomplete target 1 (IT1), three nucleotide mismatch at the 5′ end of siRNA3 guide target site.
3. Incomplete target 2 (IT2), five nucleotide mismatch at the 5′ end of siRNA3 guide target site.
4. Seed only (SO), the HBV target sequence is complementary to only the siRNA3 guide seed region.

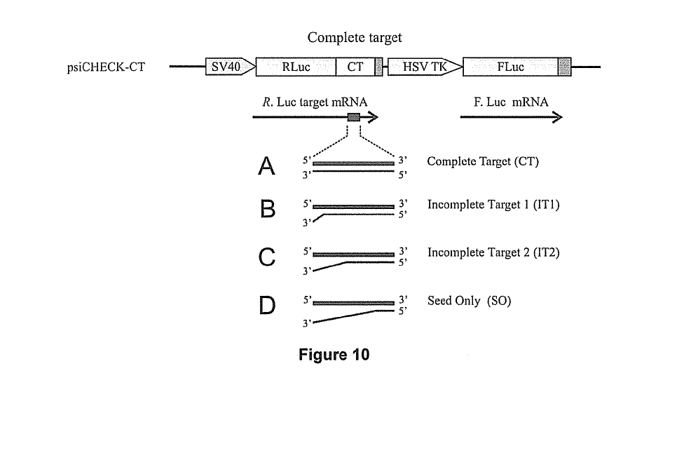

The structures of the dual luciferase reporters are illustrated schematically in FIG. 10.

The procedure for inserting CT, IT1, IT2 and SO into psiCHECK-HBx is summarised as follows, to generate the backbone for cloning of the inserts, psiCHECK-HBx (2 μg) was digested with XhoI and NotI to generate 6242 bp and 564 bp fragments. The 6242 bp psiCHECK fragment was purified from an agarose gel using a gel extraction kit (Qiagen MinElute Gel Extraction Kit), according to Manufacturer's instructions. Yield was checked after electrophoresis on a 1% agarose gel. To generate the panel of siRNA3 target sequences, the oligonucleotides listed in Table 5 below were synthesized by Integrated DNA Technologies (IDT, Iowa, USA). Twenty microliters from a 100 μM stock of each of forward and reverse oligo were combined then heated to 95° C. for 5 minutes. Thereafter, the solutions were allowed to cool to room temperature. The annealed oligonucleotides, which had sticky ends complementary to those generated by NotI and XhoI restriction digestion, were then diluted with water to a concentration of 10 mM.

TABLE 5
Sequences of oligonucleotides synthesized in order to create complete and
partial HBV targets.

| Oligonucleotide Name | Sequence |
|---|---|
| Complete target forward | 5′-TCG AGC GAC CTT GAG GCA TAC TTC AAG TCG ACC AGC |
| (CT F) | TGG C-3′ (SEQ ID NO: 37) |
| Complete target reverse | 5′-GGC CGC CAG CTG GTC GAC TTG AAG TAT GCC TCA AGG |
| (CT R) | TCG C-3′ (SEQ ID NO: 38) |
| Incomplete target 1 forward | 5′-TCG AGC GAC ACC GAG GCA TAC TTC AAG TCG ACC AGC |
| (IT1 F) | TGG C-3′ (SEQ ID NO: 39) |
| Incomplete target 1 reverse | 5′-GGC CGC CAG CTG GTC GAC TTG AAG TAT GCC TCG GTG |
| (IT1 R) | TCG C-3′ (SEQ ID NO: 40) |
| Incomplete target 2 forward | 5′-TCG AGA TCA ACC GAG GCA TAC TTC AAG TCG ACC AGC |
| (IT2 F) | TGG C-3′ (SEQ ID NO: 41) |
| Incomplete target 2 reverse | 5′-GGC CGC CAG CTG GTC GAC TTG AAG TAT GCC TCG GTT |
| (IT2 R) | GAT C-3′ (SEQ ID NO: 42) |
| Seed only target forward | 5′-TCG AGA TCA ACC ACT AAC TAC TTC AAG TCG ACC AGC |
| (SO F) | TGG C-3′ (SEQ ID NO: 43) |
| Seed only target reverse | 5′-GGC CGC CAG CTG GTC GAC TTG AAG TAG TTA GTG GTT |
| (SO R) | GAT C-3′ (SEQ ID NO: 44) |

The annealed oligonucleotides were then ligated to the digested and purified psiCHECK backbone fragment according to standard procedures. Colonies were screened by restriction digestion of isolated plasmids using PvuII. Positive clones were verified by sequencing (Inqaba Biotech, South Africa).

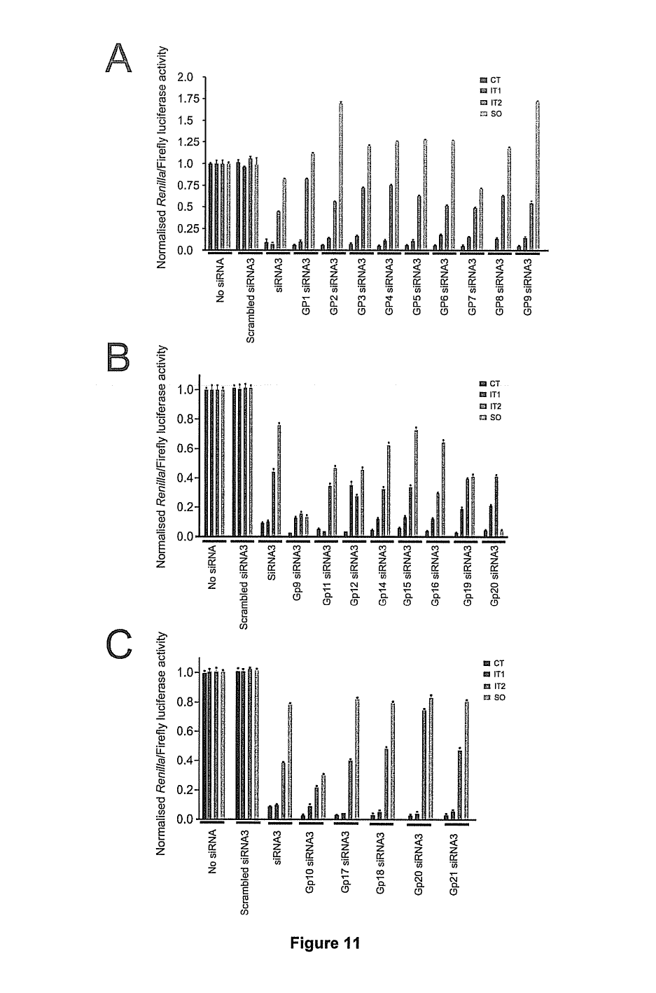

To measure knockdown efficiency of 2′-O-guanidinopropyl-modified siRNAs that were completely or partially complementary to targets, Huh7 cells were co-transfected with various unmodified or GP-containing siRNAs, together with a reporter gene plasmid (psiCHECK-CT, psiCHECK-IT1, psiCHECK-IT2, psiCHECK-SO) [20] (FIG. 11). As before, the siRNAs differed with respect to location of the 2′-O-guanidinopropyl modifications. These spanned the length of the antisense strand of the siRNA duplex, and the positioning of the modifications is indicated with respect to the 5′ end of the intended guide strand. In the reporter plasmids, the target sequences were located in the Renilla transcript but downstream of the reporter ORF (FIG. 10). Expression of Firefly luciferase is constitutively active to enable correction for variations in transfection efficiency. The ratio of Renilla to Firefly luciferase activity was used to assess knockdown efficacy and specificity of the modified siRNAs for the panel of target reporter cassettes.

Compared to a scrambled siRNA control, analysis showed that the Renilla luciferase activity was diminished by at least 85% when the reporter plasmid containing the complete target was co-transfected with the unmodified or GP-modified siRNA (FIG. 11). These results are in accordance with the previous observations carried out on the complete target within the dual luciferase reporter construct and also in the pCH-9/3091 replication competent HBV plasmid (FIGS. 5 and 6). Importantly, the knockdown of the seed only target was observed when the GP modifications were included at positions 10 to 21, which are downstream of the seed-targeting region (FIGS. 11B and 11C). Similarly, the inhibition of incomplete target 2 was more significant when the GP modifications were downstream of the seed-targeting region. Conversely, there was no observable silencing of the seed only-containing dual luciferase reporter when co-transfections were carried out with siRNAs containing GP modifications within the seed-targeting regions (FIG. 11A). This suggests that the GP modifications within the seed-targeting region diminish the interaction of the siRNA guide with an incompletely matched cognate. Importantly efficient knockdown of complete HBV target (CT) by siRNAs containing modifications within the seed-targeting region was observed. These findings indicate that siRNAs with GP modifications within the seed targeting region have improved specificity without compromised knockdown potency for HBV targets.

### Example 14

### Testing of Anti-HBV Efficacy of siRNA Sequences In Vivo Using the Hydrodynamic Injection Model of HBV Replication

Hydrodynamic Injection of Mice.

The murine hydrodynamic tail vein injection (HDI) method was employed to determine the effects of unmodified and GP-modified siRNAs on the expression of HBV genes in vivo. Experiments on animals were carried out in accordance with protocols approved by the University of the Witwatersrand Animal Ethics Screening Committee. A saline solution comprising 10% of the mouse's body mass was injected via the tail vein over 5-10 seconds. This saline solution included a combination of three plasmid vectors: 15 μg target DNA (pCH-9/3091); 25 μg anti-HBV siRNAs (unmodified siRNA3, GP3, GP4 and GP5), control non-targeting scrambled siRNA or no siRNA (saline control); and 5 μg pCl neo EGFP (a control for hepatic DNA delivery, which constitutively expresses the EGFP marker gene [32]). Each experimental group comprised 5 mice. Blood was collected under anaesthesia by retroorbital puncture on days 3 and 5 after HDI. Serum HBsAg concentration was measured using the Monolisa (ELISA) immunoassay kit (BioRad, CA, USA) according to the manufacturer's instructions. To measure effects of siRNAs on circulating viral particle equivalents (VPEs), total DNA was isolated from 50 μl of the serum of mice on days 3 and 5 after hydrodynamic injection and viral DNA determined using quantitative PCR according to previously described methods [17]. Briefly, total DNA was isolated from 50 μl of mouse serum using the Total Nucleic Acid Isolation Kit and MagNApure instrument from Roche Diagnostics. Controls included water blanks and HBV negative serum. DNA extracted from the equivalent of 8 μl of mouse serum was amplified using SYBR green Taq readymix (Sigma, Mo., USA). Crossing point analysis was used to measure virion DNA concentrations and standard curves were generated using EuroHep calibrators [33]. The HBV surface primer set was: HBV surface forward: 5′-TGC ACC TGT ATT CCA TC-3′ (SEQ ID NO: 52), and HBV surface reverse: 5′-CTG AAA GCC AAA CAG TGG-3′ (SEQ ID NO: 53). PCR was carried out using the Roche Lightcycler V.2. Capillary reaction volume was 20 μl and thermal cycling parameters consisted of a hot start for 30 sec 95° C. followed by 50 cycles of 57° C. for 10 sec, 72° C. for 7 sec and then 95° C. for 5 sec. Specificity of the PCR products was verified by melting curve analysis and agarose gel electrophoresis.

Inhibition of Markers of HBV Replication In Vivo.

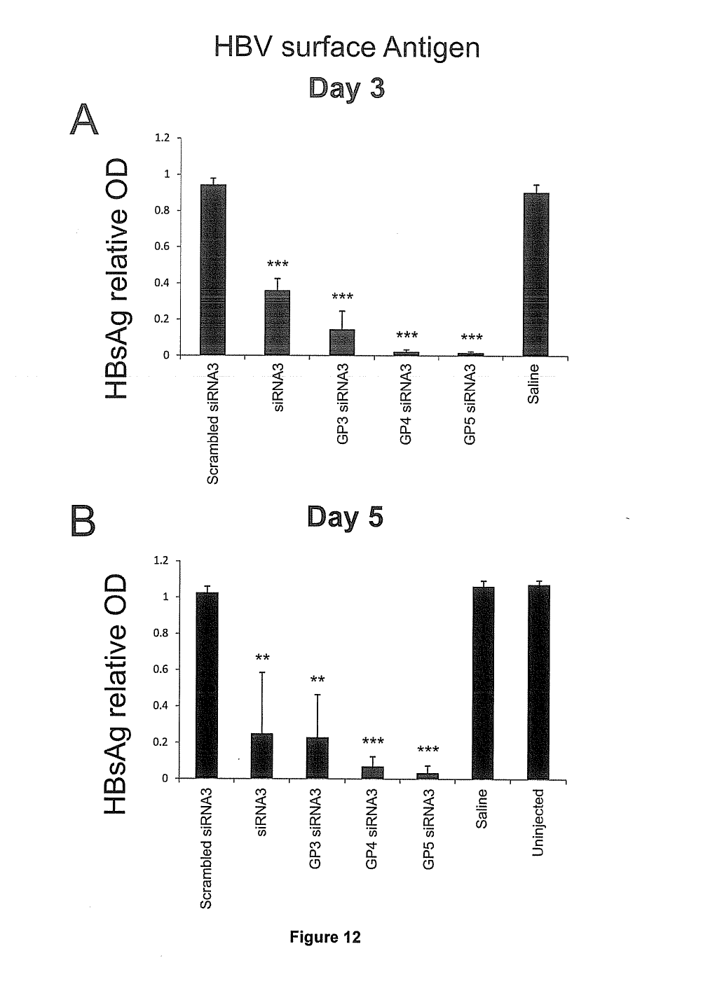

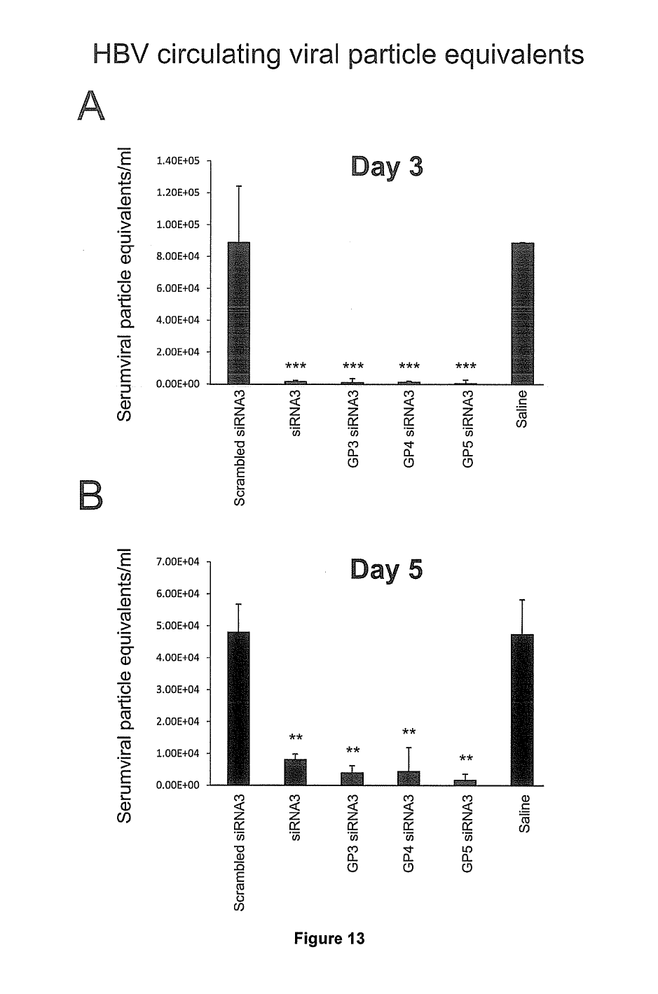

FIG. 12 shows the concentrations of HBsAg detected in the serum of mice that had been subjected to the HDI procedure with the pCH-9/3091 HBV plasmid and indicated anti-HBV and control siRNAs. The unmodified and GP-modified siRNAs each effected knockdown of the viral antigen by 70-98%. This was observed when measurements were taken at both 3 days and 5 days after HDI. Of the siRNAs, those containing GP modifications at positions 4 and 5 (GP4 and GP5) were the most efficient, and HBsAg concentration in the serum of mice injected with this plasmid was approximately 2% of the controls. The number of circulating VPEs in the same mice were also measured using quantitative real time PCR at days 3 and 5. These data are shown in FIG. 13. The results corroborate observations made on HBsAg determinations (FIG. 12) in that unmodified and GP-modified siRNAs effected highly efficient knockdown of the number of circulating VPEs. At days 3 and 5, the number of VPEs were approximately 8.9×10⁴ and 4.8×10⁴ per ml of serum respectively in the control animals. The circulating VPEs in anti-HBV siRNA-treated animals was generally more than 100-fold lower and ranged from 0.5−5×10³ per ml of serum. GP-modified and unmodified siRNAs had approximately equal efficacy in knocking down this marker of replication. Collectively, the data from FIGS. 12 and 13 show that GP-modified siRNAs are highly efficient silencers of HBV gene expression in vivo. Based on the assessment of HBsAg secretion from treated mice, the efficiency of the modified siRNAs is better than that of the unmodified siRNA3.

### Example 15

### Hybridisation Studies

The influence of 2′-O-guanidinopropyl-modified nucleosides on thermal stability of different RNA duplexes was examined. For this purpose, the G_{GP} and U_{GP} modified phosphoramidites were inserted into 12mer RNA (ON2-ON6) and the duplex melting point was measured. As shown in Table 6, the presence of 2′-O-guanidinopropyl group in oligoribonucleotides did not significantly affect the stability of duplexes, although a slight trend to destabilisation was observed. Guanidinopropyl modified building blocks gives almost the same Tm value for single, double and triple substituted oligonucleotides.

Interestingly, in one case when a 2′-O-guanidinopropyl modification of G was placed in a central position, the Tm decreased more significantly (ΔTm=−2,4° C.).

The results indicate that the thermodynamic effect of 2′-O-guanidinopropyl group is independent on the placement of the modification and which of the nucleosides is modified. After including more, but not adjacent substitutions, an additional destabilising effect was not observed (ON5 and ON6, Table 6) Moreover, for the modified oligonucleotides bearing more than one 2′-O-guanidinopropyl residue, high binding affinity to the complementary strand, was unaffected.

This observation is in accordance with the hybridisation properties of oligonucleotides containing 2′-O-aminopropyl (2′-O-AP) groups [16]. Incorporation of single 2′-O-AP units at the 3′-end or in the middle of an oligomer reduce the Tm of an RNA duplex. When adjacent residues are modified or when all nucleotides of a strand are substituted with 2′-O-AP groups duplex stabilisation occurs. Molecular dynamic and NMR data confirmed that flexibility of the aminoalkyl chain did not result in formation of strong electrostatic interactions or hydrogen bond formation [16]. Moreover, no or little stabilising effect is expected to be associated with the degree of hydration for the 2′-O-AP [22], [37].

As a result of the flexibility of the 2′-O-guanidinopropyl residue, local disruption and thermodynamic destabilisation of the modified duplexes is expected. However, presence of the guanidinium group, with three planar nitrogen atoms, allows protonation over a wide pH range. This should neutralise overall negative charge and preserve thermodynamic stability of 2′-O-guanidinopropyl-modified RNA.

TABLE 6
Effect of 2′-O-guanidinopropyl modification on duplex stability with
complementary RNA (5′-GGC AUA CUU CAA-3′) (SEQ ID NO: 45)

| Oligo | Sequence | Tₘ [° C.] | ΔTm [° C.] |
|---|---|---|---|
| ON 1 | 5′-UUG AAG UAU GCC-3′ (SEQ ID NO: 46) | 54.9 | —— |
| ON 2 | 5′-UUG_{GP} AAG UAU GCC-3′ (SEQ ID NO: 47) | 54.5 | −0.4 |
| ON 3 | 5′-UUG AAG_{GP} UAU GCC-3′ (SEQ ID NO: 48) | 52.5 | −2.4 |
| ON 4 | 5′-UUG AAG UAU_{GP} GCC-3′ (SEQ ID NO: 49) | 54.4 | −0.5 |
| ON 5 | 5′-UUG_{GP} AAG_{GP} UAU GCC-3′ (SEQ ID NO: 50) | 54.6 | −0.3 |
| ON 6 | 5′-UUG_{GP} AAG_{GP} UAU_{GP} GCC-3′ (SEQ ID NO: 51) | 54.4 | −0.5 |

TABLE 7
Effect of 2′-O-guanidinopropyl modification on duplex stability.
All Δ Tm values were measured in comparison to a control sample
with unmodified double strand in the same cuvette holder.

| | | Tm [° C.] ± | ΔTm |
|---|---|---|---|
| Antisense Oligonucleotide | Sense Oligonucleotide | 1° C. | [° C.] |
| unmodified (SEQ ID NO: 1) | unmodified (SEQ ID NO: 2) | 74 | |
| GP 4 siRNA3 (SEQ ID NO: 7) | unmodified (SEQ ID NO: 2) | 72.1 | −1.9 |
| GP 5 siRNA3 (SEQ ID NO: 8) | unmodified (SEQ ID NO: 2) | 72.9 | −1.1 |
| GP 8 siRNA3 (SEQ ID NO: 11) | unmodified (SEQ ID NO: 2) | 72.1 | −0.9 |
| GP 9 siRNA3 (SEQ ID NO: 12) | unmodified (SEQ ID NO: 2) | 72.7 | −0.9 |
| GP 11 siRNA3 (SEQ ID NO: 14) | unmodified (SEQ ID NO: 2) | 72 | −1.6 |
| GP 12 siRNA3 (SEQ ID NO: 15) | unmodified (SEQ ID NO: 2) | 72.4 | −0.6 |
| GP 14 siRNA3 (SEQ ID NO: 17) | unmodified (SEQ ID NO: 2) | 72.8 | −0.8 |
| GP 15 siRNA3 (SEQ ID NO: 18) | unmodified (SEQ ID NO: 2) | 71.3 | −1.3 |
| GP 16 siRNA3 (SEQ ID NO: 19) | unmodified (SEQ ID NO: 2) | 73.2 | −0.1 |
| GP 19 siRNA3 (SEQ ID NO: 22) | unmodified (SEQ ID NO: 2) | 72.7 | −0.4 |
| GP 20 siRNA3 (SEQ ID NO: 23) | unmodified (SEQ ID NO: 2) | 73.4 | 0.3 |
| unmodified (SEQ ID NO: 1) | S GP 17 siRNA3 (SEQ ID NO: 31) | 73 | −0.8 |
| GP 4 siRNA3 (SEQ ID NO: 7) | S GP 17 siRNA3 (SEQ ID NO: 31) | 71.5 | −2.3 |
| GP 5 siRNA3 (SEQ ID NO: 8) | S GP 17 siRNA3 (SEQ ID NO: 31) | 73 | 0.4 |
| GP 8 siRNA3 (SEQ ID NO: 11) | S GP 17 siRNA3 (SEQ ID NO: 31) | 71.7 | −2.1 |
| GP 9 siRNA3 (SEQ ID NO: 12) | S GP 17 siRNA3 (SEQ ID NO: 31) | 72.2 | −1 |
| GP 11 siRNA3 (SEQ ID NO: 14) | S GP 17 siRNA3 (SEQ ID NO: 31) | 71.8 | −1.5 |
| GP 12 siRNA3 (SEQ ID NO: 15) | S GP 17 siRNA3 (SEQ ID NO: 31) | 72.1 | −1.1 |
| GP 14 siRNA3 (SEQ ID NO: 17) | S GP 17 siRNA3 (SEQ ID NO: 31) | 72.5 | −0.7 |
| GP 15 siRNA3 (SEQ ID NO: 18) | S GP 17 siRNA3 (SEQ ID NO: 31) | 71.9 | −0.7 |
| GP 16 siRNA3 (SEQ ID NO: 19) | S GP 17 siRNA3 (SEQ ID NO: 31) | 73.2 | 0.2 |
| GP 19 siRNA3 (SEQ ID NO: 22) | S GP 17 siRNA3 (SEQ ID NO: 31) | 73.5 | 0.4 |
| GP 20 siRNA3 (SEQ ID NO: 23) | S GP 17 siRNA3 (SEQ ID NO: 31) | 73.9 | 0.8 |
| unmodified (SEQ ID NO: 1) | S GP 5, 13, 17 siRNA3 (SEQ ID NO: 32) | 73 | −1.3 |
| GP 4 siRNA3 (SEQ ID NO: 7) | S GP 5, 13, 17 siRNA3 (SEQ ID NO: 32) | 71 | −3.5 |
| GP 5 siRNA3 (SEQ ID NO: 8) | S GP 5, 13, 17 siRNA3 (SEQ ID NO: 32) | 71.5 | −3 |
| GP 8 siRNA3 (SEQ ID NO: 11) | S GP 5, 13, 17 siRNA3 (SEQ ID NO: 32) | 70.7 | −3.8 |
| GP 9 siRNA3 (SEQ ID NO: 12) | S GP 5, 13, 17 siRNA3 (SEQ ID NO: 32) | 71.2 | −2.1 |
| GP 11 siRNA3 (SEQ ID NO: 14) | S GP 5, 13, 17 siRNA3 (SEQ ID NO: 32) | 71.3 | −3.1 |
| GP 12 siRNA3 (SEQ ID NO: 15) | S GP 5, 13, 17 siRNA3 (SEQ ID NO: 32) | 70.3 | −3 |
| GP 14 siRNA3 (SEQ ID NO: 17) | S GP 5, 13, 17 siRNA3 (SEQ ID NO: 32) | 70.8 | −2.4 |
| GP 15 siRNA3 (SEQ ID NO: 18) | S GP 5, 13, 17 siRNA3 (SEQ ID NO: 32) | 70.8 | −3.7 |
| GP 16 siRNA3 (SEQ ID NO: 19) | S GP 5, 13, 17 siRNA3 (SEQ ID NO: 32) | 71.7 | −1.6 |
| GP 19 siRNA3 (SEQ ID NO: 22) | S GP 5, 13, 17 siRNA3 (SEQ ID NO: 32) | 72.4 | −0.8 |

### Example 16

### Efficacy of siRNAs Containing Single GP Modifications in the Antisense Strand and One or Three GP Modifications in the Sense Strand

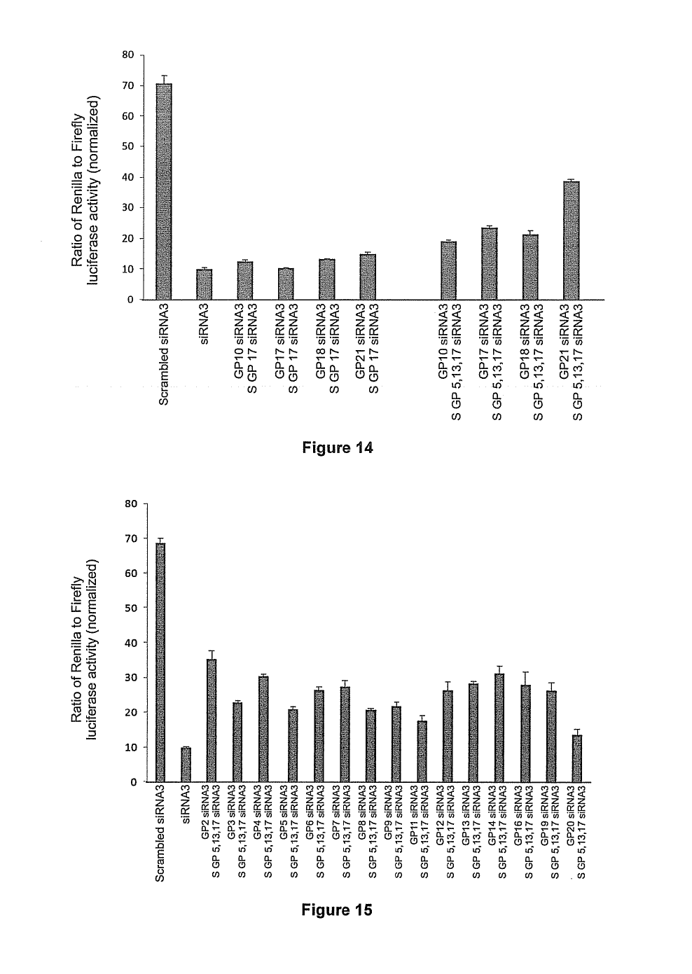

Transfections of Huh7 cells were carried out as has been described above. Briefly, 2′-O-guanidinopropyl-modified siRNAs comprising various sense and antisense combinations were used to co-transfect HEK293 cells together with a reporter gene plasmid (psiCHECK-HBx) [8] (FIGS. 14 & 15). The siRNAs targeted a single sequence of the X open reading frame (ORF) of HBV (HBx) that has previously been shown to be an effective cognate for RNAi-based silencing [9]. Each of the siRNAs differed with respect to location of the 2′-O-guanidinopropyl modification, and were positioned in the antisense and sense strands. siRNAs have been named according to the positioning of the 2′-O-guanidinopropyl (GP) modifications from the 5′ end of the antisense or sense strands. In psiCHECK-HBx, the viral target sequence was located in the Renilla transcript but downstream of the reporter ORF (FIG. 5A). Expression of Firefly lu-ciferase is constitutively active to enable correction for variations in transfection efficiency. The ratio of Renilla to Firefly luciferase activity is was used to assess knockdown efficacy.

Efficacy against the HBV targets of siRNAs comprising strands that had single modifications in both the sense or antisense strands was similar to the unmodified siRNA3 (FIG. 14), However, inclusion of three GP modifications in the sense strand and one GP modification in the antisense strand resulted in attenuated silencing efficacy (FIGS. 14 & 15). Collectively, these data reveal that although GP modifications confer favourable silencing properties on duplex siRNAs, inclusion of multiple GP residues compromises siRNA target silencing. At least one GP modification in the sense strand and one GP modification in the antisense strand does not appear to diminish siRNA3 silencing of HBV targets.

### REFERENCES

1. Haussecker, D. Hum. Gene Ther. 2008, 19, 451.
2. Tripp, R. A.; Tompkins, S. M. In Methods in Molecular Biology; Humana Press, 2009; Vol. 555, p 43.
3. Bumcrot, D.; Manoharan, M.; Koteliansky, V.; Sah, D. W. Y. Nat. Chem. Biol. 2006, 2, 711.
4. Behlke, M. A. Mol. Ther. 2006, 13, 644.
5. Behlke, M. A. Oligonucleotides 2008, 18, 305.
6. Li, J.; Huang, L. Nanomedicine 2010, 5, 1483.
7. Moschos, S. A.; Jones, S. W.; Perry, M. M.; Williams, A. E.; Erjefalt, J. S.; Turner, J. J.; Barnes, P. J.; Sproat, B. S.; Gait, M. J.; Lindsay, M. A. Bioconjugate Chem. 2007, 18, 1450.
8. Nothisen, M.; Kotera, M.; Voirin, E.; Remy, J.-S.; Behr, J.-P. J. Am. Chem. Soc. 2009, 131, 17730.
9. Engels, J. W.; Odadzic, D.; Smicius, R.; Haas, J. In Methods in Molecular Biology; Humana Press, 2010; Vol. 623, p 155.
10. Odadzic, D.; Bramsen, J. B.; Smicius, R.; Bus, C.; Kjems, J.; Engels, J. W. Bioorg. Med. Chem. 2008, 16, 518.
11. Bramsen, J. B.; Laursen, M. B.; Nielsen, A. F.; Hansen, T. B.; Bus, C.; Langkjr, N.; Babu, B. R.; Højland, T.; Abramov, M.; Van Aerschot, A.; Odadzic, D.; Smicius, R.; Haas, J.; Andree, C.; Barman, J.; Wenska, M.; Srivastava, P.; Zhou, C.; Honcharenko, D.; Hess, S.; Müller, E.; Bobkov, G. V.; Mikhailov, S, N.; Fava, E.; Meyer, T. F.; Chattopadhyaya, J.; Zerial, M.; Engels, J. W.; Herdewijn, P.; Wengel, J.; Kjems, J. Nucleic Acids Res. 2009, 37, 2867.
12. Smicius, R.; Engels, J. W. J. Org. Chem. 2008, 73, 4994.
13. Sekine, T.; Kawashima, E.; Ishido, Y. Nucleic Acids Symposium Series; Oxford University Press, 1995. p 11.
14. Sekine, M.; Satoh, T. Nucleic Acids Symposium Series; Oxford University Press: London, 1990. p 11.
15. Sekine, M.; Nakanishi, T. Nucleic Acids Symposium Series; Oxford University Press: London, 1989. p 33.
16. Haas, J.; Mueller-Kuller, T.; Klein, S.; Engels, J. W. Nucleosides, Nucleotides Nucleic Acids 2007, 26, 865.
17. Carmona, S.; Ely, A.; Crowther, C.; Moolla, N.; Salazar, F. H.; Marion, P. L.; Ferry, N.; Weinberg, M. S.; Arbuthnot, P. Mol. Ther. 2006, 13, 411.
18. Ely, A.; Naidoo, T.; Arbuthnot, P. Nucleic Acids Res. 2009, 37, e91.
19. Ely, A.; Naidoo, T.; Mufamadi, S.; Crowther, C.; Arbuthnot, P. Mol. Ther. 2008, 16, 1105.
20. Weinberg, M. S.; Ely, A.; Barichievy, S.; Crowther, C.; Mufamadi, S.; Carmona, S.; Arbuthnot, P. Mol. Ther. 2007, 15, 534.
21. Mukobata, T.; Ochi, Y.; Ito, Y.; Wada, S.; Urata, H. Bioorg. Med. Chem. Lett. 2010, 20, 129.
22. Sekine, M. J. Org. Chem. 1989, 54, 2321.
23. Saneyoshi, H.; Seio, K.; Sekine, M. J. Org. Chem. 2005, 70, 10453.
24. Haas, J.; Engels, J. W. Tetrahedron Lett. 2007, 48, 8891.
25. Feichtinger, K.; Zapf, C.; Sings, H. L.; Goodman, M. J. Org. Chem. 1998, 63, 3804.
26. Odadzic, D. Ph.D. Thesis, Goethe-University at Frankfurt am Main, August 2009.
27. Hean, J.; Crowther, C.; Ely, A.; ul Islam, R.; Barichievy, S.; Bloom, K.; Weinberg, M. S.; van Otterlo, W. A. L.; de Koning, C. B.; Salazar, F.; Marion, P.; Roesch, E. B.; LeMaitre, M.; Herdewijn, P.; Arbuthnot, P. Artif. DNA: PNA XNA 2010, 1, 17.
28. Nassal, M. J. Virol. 1992, 66, 4107.
29. Matthews, D. P.; Persichetti, R. A.; Sabol, J. S.; Stewart, K. T.; McCarthy, J. R. Nucleosides Nucleotides 1993, 12, 115.
30. Caddick, S., Judd, D. B., Lewis, A. K. d. K., Reich, M. T. & Williams, M. R. Tetrahedron 2003 59, 5417.
31. Song, E. Nat Biotechnol 2005, 23, 709.
32. Passman, M., et al Biochem Biophys Res Commun 2000, 268, 728.
33. Heerman, K. H., et al. J Clin Microbiol, 1999, 37, 68.

1. A modified short interfering RNA (siRNA) nucleic acid molecule, comprising a sense strand and an antisense strand, wherein at least one nucleotide in the sense strand or at least one nucleotide in the antisense strand is derived from a 2′-0-guanidinopropyl (GP) modified nucleoside, and wherein the modified siRNA nucleic acid molecule is capable of silencing the expression of a target sequence.

2. The modified siRNA nucleic acid molecule of claim 1, wherein the 2′-0-GP modified nucleoside is selected from the group consisting of a 2′-0-guanidinopropyl adenosine phosphoramidite, a 2′-0-guanidinopropyl cytidine phosphoramidite, a 2′-0-guanidinopropyl guanosine phosphoramidite and a 2′-0-guanidinopropyl uridine phosphoramidite or combinations thereof.

3. The modified siRNA nucleic acid molecule of claim 1, wherein the sense and antisense strands are each, independently 18 to 26 nucleotides in length.

4. The modified siRNA nucleic acid molecule of claim 3, wherein the sense and antisense strands are each 21 nucleotides in length.

5. The modified siRNA nucleic acid molecule of claim 1, wherein both the sense and antisense strands comprise artificially synthesised sequences.

6. The modified siRNA nucleic acid molecule of claim 1, wherein the antisense strand targets a complementary nucleic acid sequence of a virus.

7. The modified siRNA nucleic acid molecule of claim 1, wherein the modified siRNA nucleic acid molecule inhibits replication of a virus.

8. The modified siRNA nucleic acid molecule of claim 6, wherein the virus is a hepatitis virus.

9. The modified siRNA nucleic acid molecule of claim 8, wherein the virus is a hepatitis B virus.

10. The modified siRNA nucleic acid molecule of claim 1, wherein the modified siRNA nucleic acid molecule does not induce a detectable interferon response compared to an unmodified siRNA nucleic acid molecule when transfected into cultured cells.

11. The nucleic acid molecule of claim 1, wherein the modified siRNA nucleic acid molecule has greater stability in a standard serum assay than an unmodified siRNA nucleic acid molecule comprising the same sequence.

12. The modified siRNA nucleic acid molecule of claim 1, wherein the modified siRNA nucleic acid molecule exhibits greater knockdown of target gene expression than an unmodified siRNA nucleic acid molecule comprising the same sequence.

13. The modified siRNA nucleic acid molecule of claim 1, wherein the antisense strand comprises a sequence of SEQ ID NO: 1 and wherein the at least one nucleotide has been has been inserted at position 2, 3, 4, 5, 6, 7, 8, 9, 10, 11, 12, 13, 14, 15, 16, 17, 18, 19, 20 and/or 21 of the antisense strand.

14. The modified siRNA nucleic acid molecule of claim 1, wherein the sense strand comprises a sequence of SEQ ID NO: 2 and wherein the at least one nucleotide has been inserted at position 5, 13 and/or 17 of the sense strand.

15. A method of treatment or prevention of a viral infection, the method comprising administering a therapeutic amount of a modified siRNA nucleic acid molecule comprising
a sense strand and an antisense strand, wherein at least one nucleotide in the sense strand or at least one nucleotide in the antisense strand is a 2′-0-guanidinopropyl (GP) modified nucleoside, and wherein the modified siRNA nucleic acid molecule is capable of silencing the expression of a target sequence;
and a pharmaceutically acceptable adjuvant and/or carrier to a subject in need thereof.

16. The method of claim 15, wherein the subject is a human.

17. The method of claim 15, wherein the viral infection is hepatitis virus infection.

18. The method of claim 15, wherein the hepatitis virus infection is caused by hepatitis B.

19-26. (canceled)


SEQUENCE LISTING (53 sequences)

SEQ ID NO: 1 - RNA, 21 nt, Artificial sequence
  HBV siRNA3 antisense guide
uugaaguaugccucaaggucg

SEQ ID NO: 2 - DNA, 21 bp, Artificial sequence
  HBV siRNA3 sense strand
accuugaggcauacuucaatt

SEQ ID NO: 3 - DNA, 21 bp, Artificial sequence
  Scrambled siRNA antisense strand
uauugggugugcggucacggt

SEQ ID NO: 4 - DNA, 21 bp, Artificial sequence
  Scrambled siRNA sense strand
cgugaccgcacacccaauatt

SEQ ID NO: 5 - RNA, 21 nt, Artificial sequence
  HBV siRNA3 antisense guide modified with 2′-O-guanidinopropyl at position 2
uugaaguaugccucaaggucg

SEQ ID NO: 6 - RNA, 21 nt, Artificial sequence
  HBV siRNA3 antisense guide modified with 2′-O-guanidinopropyl at position 3
uugaaguaugccucaaggucg

SEQ ID NO: 7 - RNA, 21 nt, Artificial sequence
  HBV siRNA3 antisense guide modified with 2′-O-guanidinopropyl at position 4
uugaaguaugccucaaggucg

SEQ ID NO: 8 - RNA, 21 nt, Artificial sequence
  HBV siRNA3 antisense guide modified with 2′-O-guanidinopropyl at position 5
uugaaguaugccucaaggucg

SEQ ID NO: 9 - RNA, 21 nt, Artificial sequence
  HBV siRNA3 antisense guide modified with 2′-O-guanidinopropyl at position 6
uugaaguaugccucaaggucg

SEQ ID NO: 10 - RNA, 21 nt, Artificial sequence
  HBV siRNA3 antisense guide modified with 2′-O-guanidinopropyl at position 7
uugaaguaugccucaaggucg

SEQ ID NO: 11 - RNA, 21 nt, Artificial sequence
  HBV siRNA3 antisense guide modified with 2′-O-guanidinopropyl at position 8
uugaaguaugccucaaggucg

SEQ ID NO: 12 - RNA, 21 nt, Artificial sequence
  HBV siRNA3 antisense guide modified with 2′-O-guanidinopropyl at position 9
uugaaguaugccucaaggucg

SEQ ID NO: 13 - RNA, 21 nt, Artificial sequence
  HBV siRNA3 antisense guide modified with 2′-O-guanidinopropyl at position 10
uugaaguaugccucaaggucg

SEQ ID NO: 14 - RNA, 21 nt, Artificial sequence
  HBV siRNA3 antisense guide modified with 2′-O-guanidinopropyl at position 11
uugaaguaugccucaaggucg

SEQ ID NO: 15 - RNA, 21 nt, Artificial sequence
  HBV siRNA3 antisense guide modified with 2′-O-guanidinopropyl at position 12
uugaaguaugccucaaggucg

SEQ ID NO: 16 - RNA, 21 nt, Artificial sequence
  HBV siRNA3 antisense guide modified with 2′-O-guanidinopropyl at position 13
uugaaguaugccucaaggucg

SEQ ID NO: 17 - RNA, 21 nt, Artificial sequence
  HBV siRNA3 antisense guide modified with 2′-O-guanidinopropyl at position 14
uugaaguaugccucaaggucg

SEQ ID NO: 18 - RNA, 21 nt, Artificial sequence
  HBV siRNA3 antisense guide modified with 2′-O-guanidinopropyl at position 15
uugaaguaugccucaaggucg

SEQ ID NO: 19 - RNA, 21 nt, Artificial sequence
  HBV siRNA3 antisense guide modified with 2′-O-guanidinopropyl at position 16
uugaaguaugccucaaggucg

SEQ ID NO: 20 - RNA, 21 nt, Artificial sequence
  HBV siRNA3 antisense guide modified with 2′-O-guanidinopropyl at position 17
uugaaguaugccucaaggucg

SEQ ID NO: 21 - RNA, 21 nt, Artificial sequence
  HBV siRNA3 antisense guide modified with 2′-O-guanidinopropyl at position 18
uugaaguaugccucaaggucg

SEQ ID NO: 22 - RNA, 21 nt, Artificial sequence
  HBV siRNA3 antisense guide modified with 2′-O-guanidinopropyl at position 19
uugaaguaugccucaaggucg

SEQ ID NO: 23 - RNA, 21 nt, Artificial sequence
  HBV siRNA3 antisense guide modified with 2′-O-guanidinopropyl at position 20
uugaaguaugccucaaggucg

SEQ ID NO: 24 - RNA, 21 nt, Artificial sequence
  HBV siRNA3 antisense guide modified with 2′-O-guanidinopropyl at position 21
uugaaguaugccucaaggucg

SEQ ID NO: 25 - RNA, 21 nt, Artificial sequence
  HBV siRNA3 antisense guide modified with 2′-O-guanidinopropyl at positions 2 & 5
uugaaguaugccucaaggucg

SEQ ID NO: 26 - RNA, 21 nt, Artificial sequence
  HBV siRNA3 antisense guide modified with 2′-O-guanidinopropyl at positions 2, 5 & 17
uugaaguaugccucaaggucg

SEQ ID NO: 27 - RNA, 21 nt, Artificial sequence
  HBV siRNA3 antisense guide modified with 2′-O-guanidinopropyl at positions 2 & 3
uugaaguaugccucaaggucg

SEQ ID NO: 28 - RNA, 21 nt, Artificial sequence
  HBV siRNA3 antisense guide modified with 2′-O-guanidinopropyl at positions 2, 3 & 17
uugaaguaugccucaaggucg

SEQ ID NO: 29 - RNA, 21 nt, Artificial sequence
  HBV siRNA3 antisense guide modified with 2′-O-guanidinopropyl at positions 19 & 20
uugaaguaugccucaaggucg

SEQ ID NO: 30 - RNA, 21 nt, Artificial sequence
  HBV siRNA3 antisense guide modified with 2′-O-guanidinopropyl at positions 2, 5, 17 & 20
uugaaguaugccucaaggucg

SEQ ID NO: 31 - DNA, 21 bp, Artificial sequence
  HBV siRNA3 sense strand modified with 2′-O-guanidinopropyl at positions 17
accuugaggcauacuucaatt

SEQ ID NO: 32 - DNA, 21 bp, Artificial sequence
  HBV siRNA3 sense strand modified with 2′-O-guanidinopropyl at positions 5, 13 & 17
accuugaggcauacuucaatt

SEQ ID NO: 33 - DNA, 24 bp, Artificial sequence
  Interferon-B Forward PCR primer
tccaaattgctctcctgttgtgct

SEQ ID NO: 34 - DNA, 25 bp, Artificial sequence
  Interferon-B Reverse PCR primer
ccacaggagcttctgacactgaaaa

SEQ ID NO: 35 - DNA, 27 bp, Artificial sequence
  GAPDH Forward PCR primer
aggggtcattgatggcaacaatatcca

SEQ ID NO: 36 - DNA, 28 bp, Artificial sequence
  GAPDH Reverse PCR primer
tttaccagagttaaaagcagccctggtg

SEQ ID NO: 37 - DNA, 40 bp, Artificial sequence
  Complete Target Forward Oligonucleotide
tcgagcgaccttgaggcatacttcaagtcgaccagctggc

SEQ ID NO: 38 - DNA, 40 bp, Artificial sequence
  Complete Target Reverse Oligonucleotide
ggccgccagctggtcgacttgaagtatgcctcaaggtcgc

SEQ ID NO: 39 - DNA, 40 bp, Artificial sequence
  Incomplete Target I Forward Oligonucleotide
tcgagcgacaccgaggcatacttcaagtcgaccagctggc

SEQ ID NO: 40 - DNA, 40 bp, Artificial sequence
  Incomplete Target I Reverse Oligonucleotide
ggccgccagctggtcgacttgaagtatgcctcggtgtcgc

SEQ ID NO: 41 - DNA, 40 bp, Artificial sequence
  Incomplete Target 2 Forward Oligonucleotide
tcgagatcaaccgaggcatacttcaagtcgaccagctggc

SEQ ID NO: 42 - DNA, 40 bp, Artificial sequence
  Incomplete Target 2 Reverse Oligonucleotide
ggccgccagctggtcgacttgaagtatgcctcggttgatc

SEQ ID NO: 43 - DNA, 40 bp, Artificial sequence
  Seed Only Forward Oligonucleotide
tcgagatcaaccactaactacttcaagtcgaccagctggc

SEQ ID NO: 44 - DNA, 40 bp, Artificial sequence
  Seed Only Reverse Oligonucleotide
ggccgccagctggtcgacttgaagtagttagtggttgatc

SEQ ID NO: 45 - RNA, 12 nt, Artificial sequence
  Duplex Stability Sense Oligonucleotide
ggcauacuucaa

SEQ ID NO: 46 - RNA, 12 nt, Artificial sequence
  Duplex Stability Unmodified Antisense Oligonucleotide
uugaaguaugcc

SEQ ID NO: 47 - RNA, 12 nt, Artificial sequence
  Duplex Stability Antisense Oligonucleotide modified with 2′-O-guanidinopropyl at position 3
uugaaguaugcc

SEQ ID NO: 48 - RNA, 12 nt, Artificial sequence
  Duplex Stability Antisense Oligonucleotide modified with 2′-O-guanidinopropyl at position 6
uugaaguaugcc

SEQ ID NO: 49 - RNA, 12 nt, Artificial sequence
  Duplex Stability Antisense Oligonucleotide modified with 2′-O-guanidinopropyl at position 9
uugaaguaugcc

SEQ ID NO: 50 - RNA, 12 nt, Artificial sequence
  Duplex Stability Antisense Oligonucleotide modified with 2′-O-guanidinopropyl at positions 3 & 6
uugaaguaugcc

SEQ ID NO: 51 - RNA, 12 nt, Artificial sequence
  Duplex Stability Antisense Oligonucleotide modified with 2′-O-guanidinopropyl at positions 3, 6 & 9
uugaaguaugcc

SEQ ID NO: 52 - DNA, 17 bp, Artificial sequence
  HBV Surface Forward Primer
tgcacctgtattccatc

SEQ ID NO: 53 - DNA, 18 bp, Artificial sequence
  HBV Surface Reverse Primer
ctgaaagccaaacagtgg